(19) United States
(12) Patent Application Publication
(10) Pub. No.: US 2022/0364078 A1
(43) Pub. Date: Nov. 17, 2022

(54) MRNA LARGE SCALE SYNTHESIS AND PURIFICATION

(71) Applicants: CRISPR THERAPEUTICS AG, Zug (CH); BAYER HEALTHCARE LLC, Whippany, NJ (US)

(72) Inventors: Christopher Cheng, North Reading, MA (US); Karolina Kosakowska, Cambridge, MA (US); Caroline W. Reiss, Somerville, MA (US); Kui Wang, Wellesley, MA (US)

(21) Appl. No.: 17/741,736

(22) Filed: May 11, 2022

Related U.S. Application Data
(60) Provisional application No. 63/188,864, filed on May 14, 2021.

(51) Int. Cl. C12N 15/10; C07H 21/02

(52) U.S. Cl. CPC C12N 15/1017; C07H 21/02; C12N 15/1006

(57) ABSTRACT

Described herein is method for purifying messenger RNA (mRNA) encoding a DNA endonuclease from a sample, the method comprising: (a) loading the sample comprising the mRNA onto a monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix after one or more contaminants have been separated from the bound mRNA; and (c) separating the mRNA from dsRNA by adsorption chromatography, thereby resulting in a purified mRNA solution.

### CROSS-REFERENCE TO RELATED APPLICATIONS

The present application claims the benefit of priority of U.S. Provisional Application No. 63/188,864, filed May 14, 2021, the disclosure of which is incorporated herein by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “CB36_Seqlisting.txt”, which was created on May 11, 2022 and is 67,446 bytes in size. The subject matter of the Sequence Listing is incorporated herein in its entirety by reference.

### BACKGROUND

Traditionally, mRNA is purified from in vitro transcription reactions by either commercially-available silica-based column systems, such as the Qiagen RNeasy® kit, or by protein extraction into an organic mix (phenol:chloroform:isoamyl alcohol) and subsequent ethanol precipitation. These methods are limited in scale as they can provide maximally five to ten mg of clean and homogeneous mRNA; thus, they are inadequate for the needs of clinical and commercial uses of mRNA. Tangential flow filtration (TFF) has been modified to purify precipitated mRNA from in vitro transcription reactions, which has greatly increased the scale of purification, but a need still exists for a method that produces large-scale purified mRNA compositions, e.g., that are usable in purifying an mRNA therapeutic such as an mRNA replacement therapeutic.

### SUMMARY

In one aspect, described herein is a method for purifying messenger RNA (mRNA) encoding a DNA endonuclease from a sample, the method comprising: (a) loading the sample comprising the mRNA onto a monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix after one or more contaminants have been separated from the bound mRNA; and (c) separating the mRNA from dsRNA by adsorption chromatography, thereby resulting in a purified mRNA solution.

In another aspect, described herein is a method for separating double stranded RNA (dsRNA) from mRNA encoding a DNA endonuclease, the method comprising: (a) loading a sample comprising the mRNA with monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix, thereby resulting in a semi-purified mRNA solution; and; (c) separating the mRNA in the semi-purified mRNA solution from dsRNA by adsorption chromatography, thereby resulting in a purified mRNA solution.

In another aspect, described herein is A method for producing purified mRNA encoding a DNA endonuclease, comprising: (a) linearizing a codon optimized DNA plasmid encoding the endonuclease; (b) subjecting the plasmid of (a) to an IVT reaction in the presence of a modified uridine nucleotide to synthesize mRNA comprising the modified uridine nucleotide; (c) purifying the mRNA by a method comprising: (i) loading the sample comprising the mRNA onto a monolithic matrix comprising a poly(dT) or poly(U) nucleic acid linked/coupled to the monolithic matrix such that the mRNA binds the column, wherein the mRNA comprises the nucleotide sequence of SEQ ID NO: 2, and wherein uridines in the mRNA are replaced with N-1-methylpseudouridine; (ii) eluting the mRNA from the column after one or more contaminants have been separated from the bound mRNA; and (iii) separating the mRNA of (b) from dsRNA by adsorption chromatography; thereby producing a purified mRNA solution.

In another aspect, described herein is a kit comprising a monolithic matrix comprising a ligand comprising: i) a reactive moiety coupled to the monolithic matrix, and ii) a ligand that binds to an mRNA; and instructions for purifying a sample comprising an mRNA of interest using the monolithic matrix followed by adsorption chromatography to separate dsRNA from the mRNA of interest.

In another aspect, described herein is a kit comprising a substrate for adsorption chromatography, and instructions for removing dsRNA from a solution comprising an mRNA of interest, wherein the solution was previously purified with a monolithic matrix comprising a ligand comprising: i) a reactive moiety coupled to the monolithic matrix, and ii) a ligand that binds to the mRNA.

In another aspect, described herein is a kit comprising: a monolithic matrix comprising a ligand comprising i) a reactive moiety coupled to the monolithic matrix, and ii) a ligand that binds to an mRNA; a substrate for adsorption chromatography; and instructions for purifying a sample comprising an mRNA of interest using the monolithic matrix followed by adsorption chromatography to separate dsRNA from the mRNA of interest.

### BRIEF DESCRIPTION OF THE FIGURES

FIG. 1 provides ion pair reverse phase HPLC chromatograms of 10 μg oligo dT purified mRNA with various percentages of Buffer B (“B44” is 44% Buffer B, “B54” is 54% Buffer B, “B49” is 49% Buffer B, “B45” is 45% Buffer B, and “B39” is 39% Buffer B) at 25° C. (“25 C”) or 45° C. (“45 C”).

FIG. 2 provides ion pair reverse phase HPLC chromatograms of various loading amounts of oligo dT purified mRNA at 45% Buffer B.

FIG. 3 provides ion pair reverse phase HPLC chromatograms of 100 oligo dT purified mRNA with various percentages of Buffer B (“B40” is 40% Buffer B, “B42” is 42% Buffer B, “B45” is 45% Buffer B, “B50” is 50% Buffer B, and “B55” is 55% Buffer B) at 25° C. (“25 C”) or 45° C. (“45 C”).

### DETAILED DESCRIPTION

The present disclosure provides large scale synthesis and purification of mRNAs encoding an S. pyogenes Cas9 endonuclease (“SpCas9 mRNA”), and which optionally include chemically modified nucleotides, that provide effective genome editing of a target cell population when administered with one or more gRNAs. The methods of synthesis and purification described herein produces mRNA with reduced immunogenicity compared to mRNA produced according to other purification methods known in the art. Given that SpCas9 mRNA is a large, complex biomolecule the purification is likewise complicated as numerous impurities are present in the crude product. By breaking the purification into two step, the described protocol enables for a preliminary ‘normalization’ of impurities, which better enables their removal from the final, pure SpCas9 mRNA product.

mRNA Synthesis

The methods described herein may be used to purify any mRNA. mRNA is typically thought of as the type of RNA that carries information from DNA to the ribosome. The existence of mRNA is typically very brief and includes processing and translation, followed by degradation. Typically, in eukaryotic organisms, mRNA processing comprises the addition of a “cap” on the N-terminal (5′) end, and a “tail” on the C-terminal (3′) end. A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. The tail is typically a polyadenylation event whereby a polyadenylyl moiety is added to the 3′ end of the mRNA molecule. The presence of this “tail” serves to protect the mRNA from exonuclease degradation. Messenger RNA is translated by the ribosomes into a series of amino acids that make up a protein.

mRNAs according to the present disclosure may be synthesized according to any of a variety of known methods, including but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. For example, mRNAs according to the present disclosure may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The presence of these reagents is undesirable in the final product according to several embodiments and may thus be referred to as impurities and a preparation containing one or more of these impurities may be referred to as an impure preparation.

According to various embodiments, the methods described herein are used to purify in vitro synthesized mRNA of a variety of lengths. In some embodiments, the methods described herein are used to purify in vitro synthesized mRNA of or greater than about 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, or 20 kb in length. In some embodiments, the methods described herein are used to purify in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length. For example, typical mRNAs may be about 1 kb to about 5 kb in length. More typically, the mRNA will have a length of about 1 kb to about 3 kb. However, in some embodiments, the mRNA in the composition is much longer (greater than about 20 kb). In some embodiments, the methods described herein are used to purify mRNA containing one or more modifications that typically enhance stability. In some embodiments, one or more modifications are selected from modified nucleotide, modified sugar phosphate backbones, 5′ and/or 3′ untranslated region. In some embodiments, the methods described herein are used to purify in vitro synthesized mRNA that is unmodified.

Typically, mRNAs are modified to enhance stability. Modifications of mRNA can include, for example, modifications of the nucleotides of the RNA. A modified mRNA according to the invention can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

In some embodiments, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

In some embodiments, the at least one modification provides (i) improved mRNA stability, for example, in serum or in cells, (ii) improved mRNA translation efficiency, and/or (iii) reduced activation of innate immune signaling pathways compared to an equivalent unmodified mRNA. In some embodiments, the at least one modification improves the level and/or duration of expression of the encoded site-directed endonuclease, such as SpCas9 polypeptide, in a target tissue or cell following systemic administration of the mRNA (e.g., as compared to an equivalent unmodified mRNA). In some embodiments, the at least one modification reduces activation of innate immune cell responses following systemic administration of the mRNA (e.g., as compared to an equivalent unmodified mRNA).

In some embodiments, the at least one modification is selected from: (i) sequence optimization of the mRNA, (ii) chemical modification of at least one nucleotide of the mRNA, or (iii) a combination of (i) and (ii).

In some embodiments, the mRNA purified according to the methods disclosed herein comprises a sequence-optimized nucleotide sequence. In some embodiments, the mRNA comprises a nucleotide sequence that is sequence optimized for expression in a target cell. In some embodiments, the target cell is a mammalian cell. In some embodiments, the target cell is a human cell, a murine cell, or a non-human primate (NHP) cell.

A sequence-optimized nucleotide sequence, e.g., a codon-optimized mRNA sequence encoding a site-directed endonuclease, such as a SpCas9 polypeptide, typically is a sequence comprising at least one synonymous nucleobase substitution with respect to a reference sequence (e.g., a non-optimized mRNA sequence encoding a site-directed endonuclease, such as a SpCas9 polypeptide). A sequence-optimized nucleotide sequence can be partially or completely different in sequence from the reference sequence. For example, a reference sequence encoding polyserine uniformly encoded by TCT codons can be sequence-optimized by having 100% of its nucleobases substituted (for each codon, T in position 1 replaced by A, C in position 2 replaced by G, and T in position 3 replaced by C) to yield a sequence encoding polyserine which would be uniformly encoded by AGC codons. The percentage of sequence identity obtained from a global pairwise alignment between the reference polyserine nucleic acid sequence and the sequence-optimized polyserine nucleic acid sequence would be 0%. However, the protein products from both sequences would be 100% identical.

Some sequence optimization (also sometimes referred to as codon optimization) methods are known in the art and can be useful to achieve one or more desired results. These results can include, e.g., matching codon frequencies in certain tissue targets and/or host organisms to ensure proper folding; uridine depletion; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide.

In some embodiments, the mRNA purified according to the methods disclosed herein comprises a nucleotide sequence that is sequence-optimized relative to a reference sequence using a method of sequence optimization. Methods of sequence optimization are known in the art, and include known sequence optimization tools, algorithms and services. Non-limiting examples include services from GeneArt (Life Technologies), DNA2.0 (Menlo Park Calif.), Geneious®, GeneGPS® (Atum, Newark, Calif.), and/or proprietary methods. In some embodiments, the purified mRNA comprises a nucleotide sequence that is sequence-optimized relative to a reference sequence using a method of sequence optimization (e.g., GeneGPS®, e.g., Geneious®). In some embodiments, the method of sequence optimization comprises any one codon optimization algorithm described in U.S. Pat. Nos. 7,561,972; 7,561,973; 8,126,653; and 8,401,798, each of which is incorporated herein by reference. In some embodiments, the nucleotide sequence is (i) sequence-optimized based on codon usage bias in a host cell (e.g., mammalian cell, e.g., human cell, murine cell, non-human primate cell) relative to a reference sequence, (ii) uridine-depleted relative to a reference sequence, or (iii) a combination of (i) and (ii), using a method of sequence optimization (e.g., GeneGPS®, e.g., Geneious®).

In some embodiments, the reference sequence comprises the nucleotide sequence of SEQ ID NO: 17. In some embodiments, the sequence-optimized nucleotide sequence comprises one or more nucleobase substitutions relative to the reference sequence. In some embodiments, the sequence-optimized nucleotide sequence is less than about 95%, about 94%, about 93%, about 92%, about 91%, about 90%, about 89%, about 88%, about 87%, about 86%, about 85%, about 84%, about 83%, about 82%, about 81%, or about 80% identical to the reference sequence. In some embodiments, the sequence-optimized nucleotide sequence is at least about 80%, at least about 81%, at least about 82%, at least about 83%, at least about 84%, at least about 85%, at least about 86%, at least about 87%, at least about 88%, at least about 89, at least about 90%, at least about 91%, at least about 92%, at least about 93%, at least about 94%, at least about 95%, at least about 96%, at least about 97%, at least about 98%, or at least about 99% identical to the reference sequence. In some embodiments, the polypeptide encoded by the sequence-optimized nucleotide sequence is 100% identical to the polypeptide encoded by the reference sequence. In some embodiments, the polypeptide encoded by the sequence-optimized nucleotide sequence is set forth in SEQ ID NO: 5.

In some embodiments, the sequence-optimized nucleotide sequence is uridine depleted, e.g., compared to the reference sequence, e.g., compared to the nucleotide sequence of SEQ ID NO: 17. In some embodiments, the uracil content of the sequence-optimized nucleotide sequence is decreased (e.g., by about 1.1-fold, 1.2-fold, 1.3-fold, 1.4-fold, or about 1.5-fold) compared to the reference sequence, e.g., the nucleotide sequence of SEQ ID NO: 17.

In some embodiments, the sequence-optimized nucleotide sequence is not uridine depleted, e.g., compared to the reference sequence, e.g., compared to the nucleotide sequence of SEQ ID NO: 17. In some embodiments, the uracil content of the sequence-optimized nucleotide sequence is substantially equivalent (e.g., about 95% to about 105% similar) or increased (e.g., by about 1.1-fold, about 1.2-fold, about 1.3-fold, about 1.4-fold, or about 1.5-fold) compared to the reference sequence, e.g., the nucleotide sequence of SEQ ID NO: 17.

In some embodiments, the mRNA purified according to the methods disclosed herein comprises a sequence-optimized nucleotide sequence, wherein the mRNA has one or more improved properties (e.g., compared to an mRNA comprising the reference sequence, e.g., compared to an mRNA comprising the nucleotide sequence of SEQ ID NO: 17). In some embodiments, the one or more improved properties relates to expression efficacy after administration in vivo. In some embodiments, the one or more improved properties include, but are not limited to, increased cutting efficiency and/or activity, improving mRNA stability, increasing translation efficacy in the target tissue or target cell, reducing the number of truncated proteins expressed, improving folding or prevent misfolding of the expressed proteins, reducing toxicity of the expressed products, reducing cell death caused by the expressed products, increasing and/or decreasing protein aggregation, or a combination thereof.

In some embodiments, the sequence-optimized nucleotide sequence is codon optimized for expression in human subjects, having structural and/or chemical features that avoid or reduce one or more of the problems known in the art, for example, features that are useful for optimizing formulation and delivery of mRNA-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

In some embodiments, the disclosure provides purified mRNAs with chemistries suitable for delivery, tolerability, and stability within cells, e.g., following in vivo or in vitro administration. Accordingly, in some embodiments, mRNAs described herein are modified, e.g., comprise a modified sugar moiety, a modified internucleoside linkage, a modified nucleoside, a modified nucleotide and/or combinations thereof. In some embodiments, the modified mRNAs exhibit one or more of the following properties: are not immune stimulatory; are nuclease resistant; have improved cell uptake; have increased half-life; have increased translation efficiency; and/or are not toxic to cells or mammals, e.g., following contact with cells in vivo or ex vivo or in vitro.

Additionally, certain nucleotide and nucleoside modifications have been shown to reduce immune stimulation, e.g., stimulation of innate immune pathways, by exogenous mRNA (see, e.g., Kariko, K, et al (2005) IMMUNITY 23:165; Anderson, et al (2011) NUCLEIC ACIDS RES 39:9329; Warren et al (2010) CELL STEM CELL 7:618).

Accordingly, the disclosure provides mRNA comprising chemical modification of one or more nucleosides/nucleotides. In some embodiments, one or more uridines of the mRNA are chemically-modified or replaced with a chemically-modified nucleoside. In some embodiments, the chemically-modified nucleoside selected from: pseudouridine, N1-methylpseudouridine, and 5-methoxyuridine. In some embodiments, the chemically-modified nucleoside is any one described in WO/2017/181107, WO/2018/144775, or WO/2020/056304, each of which is incorporated by reference herein.

In some embodiments, about 100% of the uridines of the mRNA are chemically-modified. In some embodiments, about 95% of the uridines of the mRNA are chemically-modified. In some embodiments, about 90% of the uridines of the mRNA are chemically-modified. In some embodiments, about 85% of the uridines of the mRNA are chemically-modified. In some embodiments, about 80% of the uridines of the mRNA are chemically-modified.

In some embodiments, about 100% of the uridines of the mRNA are chemically-modified and/or replaced with N-1-methylpseudouridine. In some embodiments, about 95% of the uridines of the mRNA are chemically-modified and/or replaced with N-1-methylpseudouridine. In some embodiments, about 90% of the uridines of the mRNA are chemically-modified and/or replaced with N-1-methylpseudouridine. In some embodiments, about 85% of the uridines of the mRNA are chemically-modified and/or replaced with N-1-methylpseudouridine. In some embodiments, about 80% of the uridines of the mRNA are chemically-modified and/or replaced with N-1-methylpseudouridine.

In some embodiments, the modified nucleobase is N-1-methylpseudouridine, and the mRNA of the disclosure is fully modified with N-1-methylpseudouridine. In some embodiments, N-1-methylpseudouridine represents from 75-100% of the uracils in the mRNA. In some embodiments, N-1-methylpseudouridine represents 100% of the uracils in the mRNA.

In some embodiments, the mRNA purified according to the methods disclosed herein is modified in the coding region (e.g., an open reading frame encoding a site-directed endonuclease, such as a SpCas9 polypeptide). In some embodiments, the mRNA is modified in regions besides a coding region. For example, in some embodiments, a 5′ UTR and/or a 3′ UTR are provided, wherein either or both may independently contain one or more different nucleoside modifications. In such embodiments, nucleoside modifications may also be present in the coding region.

mRNA Purification

A purification process according to present disclosure can be carried out during or subsequent to synthesis. For example, mRNA may be purified as described herein before a cap and/or tail are added to the mRNA. In some embodiments, the mRNA is purified after a cap and/or tail are added to the mRNA. In some embodiments, the mRNA is purified after a cap is added. In some embodiments, the mRNA is purified both before and after a cap and/or tail are added to the mRNA. In general, a purification step as described herein may be performed after each step of mRNA synthesis, optionally along with other purification processes, such as dialysis.

In some embodiments, the methods described herein are used to purify a single mRNA species, i.e. the mRNA preparation to be purified contains mRNA derived from a single gene or a single synthesis or expression construct. In contrast, total mRNA purified from a cell contains multiple mRNA species.

In one aspect, described herein are methods for purifying mRNA encoding a DNA endonuclease (e.g., SpCas9) from a sample comprising (a) loading the sample comprising the mRNA onto a monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix after one or more contaminants have been separated from the bound mRNA; and (c) separating the mRNA from dsRNA by adsorption chromatography, thereby resulting in a purified mRNA solution. In some embodiments, the adsorption chromatography comprises a liquid mobile phase and a solid stationary support. In some embodiments, the stationary support is non-polar.

In some embodiments, the adsorption chromatography is reverse phase liquid chromatography. Reverse phase high-performance liquid chromatography (RP-HPLC) uses a non-polar stationary phase and a moderately polar mobile phase and therefore works with hydrophobic interactions which result from repulsive forces between a relatively polar solvent, the relatively non-polar analyte, and the non-polar stationary phase (reversed phase principle). The retention time on the column is therefore longer for molecules which are more non-polar in nature, allowing polar molecules to elute more readily. The retention time is increased by the addition of polar solvent to the mobile phase and decreased by the addition of more hydrophobic solvent.

In some embodiments, the sample is loaded onto the column for reverse phase chromatography and the elution buffer is about 35% to about 55% Buffer B, optionally about 50% Buffer B, and the remainder comprising Buffer A, wherein Buffer A comprises 0.1M TEAA and Buffer B comprises 0.1M TEAA and 25% acetonitrile.

In some embodiments, the flow rate through the column is about 0.5 mL/min-5.0 mL/min, optionally about 3 mL/min.

In some embodiments, the mRNA is loaded onto the column for reverse phase chromatography at a concentration of 0.05-5.00 mg/mL, optionally 0.20-0.40 mg/mL.

In some embodiments, the purified mRNA solution is further processed to exchange the buffer. In some embodiments, the buffer is exchanged by a tangential flow filtration (TFF) system.

In another aspect, described herein are methods for separating double stranded RNA (dsRNA) from mRNA encoding a DNA endonuclease (e.g., SpCas9) comprising (a) loading a sample comprising the mRNA with monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix, thereby resulting in a semi-purified mRNA solution; and; (c) separating the mRNA in the semi-purified mRNA solution from dsRNA by adsorption chromatography, thereby resulting in a purified mRNA solution.

In another aspect, described herein are methods for producing purified mRNA encoding a DNA endonuclease (e.g., SpCas9) comprising (a) linearizing a codon optimized DNA plasmid encoding the endonuclease; (b) subjecting the plasmid of (a) to an IVT reaction in the presence of a modified uridine nucleotide to synthesize mRNA comprising the modified uridine nucleotide; and (c) purifying the synthesized mRNA comprising the modified uridine nucleotide. In some embodiments, the purifying comprises loading the synthesized mRNA onto a monolithic matrix comprising a poly(dT) or poly(U) nucleic acid molecule linked/coupled to the monolithic matrix under conditions allowing the mRNA to hybridize with the poly(dT) or poly(U) nucleic acid molecule; eluting the mRNA from the monolith matrix after one or more contaminants have been separated from the bound mRNA. In some embodiments, the method further comprises separating the mRNA in the semi-purified mRNA solution from dsRNA by adsorption chromatography.

In some embodiments, the synthesized mRNA is loaded onto a monolithic matrix. A “monolithic matrix” refers to a continuous bed consisting of a single piece of a highly porous solid material where the pores are highly interconnected forming a network of flow-through channels. The void volume is decreased to a minimum and all the mobile phase is forced to flow through the large pores of the medium. Three types of monolithic matrices are commercially available:

1) Silica gel based monolithic beds which are solid rods of silica monolith that have been prepared according to a sol-gel process. This process is based on the hydrolysis and polycondensation of alkoxysilanes in the presence of water-soluble polymers. The method leads to “rods” made of a single piece of porous silica with a defined bimodal pore structure having macro (of about 2 μm) and mesopores (of about 0.013 μm) when smaller rods intended for analytical purposes are prepared. 2) Polyacrylamide based monolithic beds are made of swollen polyacrylamide gel compressed in the shape of columns. Their technology relies on the polymerization of advanced monomers and ionomers directly in the chromatographic column. In the presence of salt, the polymer chains form aggregates into large bundles by hydrophobic interaction, creating voids between the bundles (irregularly shaped channels) large enough to permit a high hydrodynamic flow. 3) Rigid organic gel based monolithic beds: These supports are prepared by free radical polymerization of a mixture of a polymerizable monomer, optionally with functional groups, such as glycidyl methacrylate, ethylene dimethacrylate, a crosslinking agent, a radical chain initiator, such as 2,2′-azobisisobutyronitrile, and porogenic solvents (cyclohexanol and dodecanol) in barrels of an appropriate mold (Svec F, Tennikova T B (1991) J Bioact Compat Polym 6: 393; Svec F, Jelinkova M, Votavova E (1991) Angew Macromol Chem 188: 167; Svec F, Frechet J M J (1992) Anal Chem 64: 820) in the case of glycidyl methacrylate-co-ethylene dimethacrylate (GMA-EDMA) monoliths.

In various embodiments, the monolithic matrix comprises a poly(dT) or poly(U) nucleic acid molecule linked/coupled to the monolithic matrix under conditions allowing the mRNA to hybridize with the poly(dT) or poly(U) nucleic acid molecule (i.e., an oligoDT column).

In some embodiments, the one or more contaminants are selected from the group of proteins, unreacted nucleotides, plasmid DNA, CAP analogues, partial transcripts, dsRNA side products and enzymes.

In some embodiments, non-natural modified nucleobases are introduced into a mRNA during synthesis or post-synthesis. In certain embodiments, modifications are on internucleoside linkages, purine or pyrimidine bases, or sugar. In particular embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some embodiments, the mRNA solution purified according to the methods disclosed herein has reduced immunogenicity compared to mRNA purified via step (a) and not via adsorption chromatography. The phrase “reduced immunogenicity” as used herein refers to a reduction of in serum levels of MCP1, IL-6 or both.

Characterization of Purified mRNA

A particular advantage provided by the methods disclosed herein is the ability to purify mRNA, in particular, mRNA synthesized in vitro, at a large or commercial scale. For example, in vitro synthesized mRNA may be purified at a scale of or greater than about 1 gram, 10 gram, 50 gram, 100 gram, 200 gram, 300 gram, 400 gram, 500 gram, 600 gram, 700 gram, 800 gram, 900 gram, 1 kg, 5 kg, 10 kg, 50 kg, or 100 kg per batch.

In various embodiments, mRNA purified according to the present invention is substantially free of impurities from mRNA synthesis process including, but not limited to, prematurely aborted RNA sequences, DNA templates, and/or enzyme reagents used in in vitro synthesis.

Yield, capillary electrophoresis and dsRNA ELISA analysis can be performed to evaluate the integrity of the mRNA produced as described in the Examples.

In some embodiments, the mRNA solution purified according to the methods disclosed herein has less than 0.015% dsRNA (e.g., less than 0.014%, less than 0.013%, less than 0.012%, less than 0.011%, less than 0.001%). In some embodiments, the dsRNA is not detectable in the purified mRNA solution.

Messenger RNAs Encoding a Site-Directed Endonuclease

In some aspects, the disclosure provides a purified mRNA encoding a site-directed endonuclease, such as a SpCas9 polypeptide, for use in methods of genome editing using a CRISPR/Cas system. In some embodiments, the mRNA comprises a 5′ UTR, an open reading frame (ORF) comprising a nucleotide sequence encoding a site-directed endonuclease, such as a SpCas9 polypeptide, and a 3′ UTR.

In some embodiments, the mRNA purified according to the methods disclosed herein comprises an open-reading frame (ORF), wherein the ORF comprises a nucleotide sequence that encodes a site-directed endonuclease, such as a Cas nuclease, wherein the Cas nuclease is a SpCas9 polypeptide. In some embodiments, the Cas nuclease comprises at least one domain that interacts with a guide RNA (gRNA). Additionally, the Cas nuclease is directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the mRNA purified according to the methods disclosed herein comprises a 5′ untranslated region (5′ UTR), a 3′ untranslated region (3′ UTR), and an ORF comprising a nucleotide sequence encoding a site-directed endonuclease, such as a SpCas9 polypeptide. In some embodiments, the mRNA further comprises a 5′ cap structure, a Kozak or Kozak-like sequence (also known as a Kozak consensus sequence), a polyA sequence (also known as a polyadenylation signal), a nucleotide sequence encoding a nuclear localization signal (NLS), a nucleotide sequence encoding a linker peptide, a nucleotide sequence encoding a tag peptide, or any combination thereof. In some embodiments, the consensus Kozak consensus sequence facilitates the initial binding of mRNA to ribosomes, thereby enhances its translation into a polypeptide product.

In some embodiments, the mRNA purified according to the methods disclosed herein comprises any suitable number of base pairs, e.g., thousands (e.g., 4000, 5000, 6000, 7000, 8000, 9000, or 10,000) of base pairs. In some embodiments, the mRNA is about 4.2 kb, about 4.3 kb, about 4.4 kb, about 4.5 kb, about 4.6 kb, about 4.7 kb, about 4.8 kb, about 4.9 kb, about 5.0 kb, about 5.1 kb, about 5.2 kb, about 5.3 kb, about 5.4 kb, about 5.5 kb, or more in length.

A. 5′ and 3′ Untranslated Regions (UTRs)

In some embodiments, the 5′ UTR or 3′ UTR is derived from a human gene sequence. Non-limiting exemplary 5′ UTR and 3′ UTR include those derived from genes encoding a- and β-globin, albumin, HSD17B4, and eukaryotic elongation factor 1a. In addition, viral-derived 5′ UTR and 3′ UTRs can also be used and include orthopoxvirus and cytomegalovirus UTR sequences.

B. 5′Cap

In some embodiments, the mRNA purified according to the methods disclosed herein comprises a 5′ cap structure. A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. This cap is a cap-0 where nucleotide N does not contain 2′OMe, or cap-1 where nucleotide N contains 2′OMe, or cap-2 where nucleotides N and N+1 contain 2′OMe. This cap may also be of the structure m2 7′3″G(5′)N as incorporated by the anti-reverse-cap analog (ARCA), and may also include similar cap-0, cap-1, and cap-2, etc., structures.

In some embodiments, the 5′cap is a CleanCap® (TriLink Biotechnologies) capping structure. Non-limiting examples of CleanCap® capping structures include CleanCap® Reagent GG (m7G(5′)ppp(5′)(2′OMeG)pG, CleanCap® Reagent AU (m7G(5′)ppp(5′)(2′OMeA)pU, and CleanCap® Reagent AG (m7(3′OMeG)(5′)ppp(5′)(2′OMeA)pG,

In some embodiments, the 5′ cap may regulate nuclear export; prevent degradation by exonucleases; promote translation; and promote 5′ proximal intron excision. Stabilizing elements for caps include phosphorothioate linkages, boranophosphate modifications, and methylene bridges. In addition, caps may also contain a non-nucleic acid entity that acts as the binding element for eukaryotic translation initiation factor 4E, eIF4E.

C. Nuclear Localization Signal

In some embodiments, the mRNA purified according to the methods disclosed herein further comprises a nucleotide sequence encoding a nuclear localization signal (NLS). In some embodiments, the nuclease is fused with more than one NLS. In some embodiments, one or more NLS is operably-linked to the N-terminus, C-terminus, or both, of the site-directed endonuclease, optionally via a peptide linker. In some embodiments, the NLS comprises a nucleoplasmin NLS and/or a SV40 NLS. In some embodiments, the mRNA comprises a nucleotide sequence encoding a nucleoplasmin NLS and a nucleotide sequence encoding an SV40 NLS.

D. Poly-A Tail

In some embodiments, the mRNA purified according to the methods disclosed herein comprises a poly(A) tail (i.e., polyA sequence, i.e., polyadenylation signal). In some embodiments, the polyA sequence comprises entirely or mostly of adenine nucleotides or analogs or derivatives thereof. In some embodiments, the polyA sequence is a tail located adjacent (e.g., towards the 3′ end) of a 3′ UTR of an mRNA. In some embodiments, the polyA sequence promotes or increases the nuclear export, translation, and/or stability of the mRNA.

In some embodiments, the poly(A) tail is about 40 to about 300 nucleotides in length. In some embodiments, the tail is about 40 to about 100 nucleotides in length. In some embodiments, the tail is about 100 to about 300 nucleotides in length. In some embodiments, the tail is about 100 to about 200 nucleotides in length. In some embodiments, the tail is about 50 to about 200 nucleotides in length. In some embodiments, the tail is about 50 to about 250 nucleotides in length. In some embodiments, the tail is about 100, 110, 120, 130, 140, 150, 160, 170, 180, 190, or 200 nucleotides in length. In some embodiments, the poly(A) tail comprises modifications to prevent exonuclease degradation, including phosphorotioate linkages and modifications to the nucleobase.

In some embodiments, the poly(A) tail comprises a 3′ “cap” comprising modified or non-natural nucleobases or other synthetic moieties.

In some embodiments, the mRNA purified according to the methods disclosed herein comprises (i) a 5′ untranslated region (UTR); (ii) an open reading frame (ORF) comprising a nucleotide sequence that encodes a site-directed endonuclease; and (iii) a 3′ untranslated region (UTR). In some embodiments, the site-directed endonuclease is a Cas nuclease. In some embodiments, the Cas nuclease is a Cas9 polypeptide. In some embodiments, the Cas 9 polypeptide is a Streptococcus pyogenes-derived Cas9 (SpCas9) polypeptide. In some embodiments, the ORF further comprises one or more nucleotide sequences encoding a nuclear localization signal, such as one described herein. In some embodiments, the ORF comprises a nucleotide sequence encoding a site-directed endonuclease, such as a SpCas9 polypeptide and at least one NLS that is a nucleoplasmin and/or SV40 NLS. In some embodiments, the ORF comprises a nucleotide sequence encoding an N-terminal and/or C-terminal NLS operably-linked to a site-directed endonuclease, such as a SpCas9 polypeptide. In some embodiments the ORF comprises a nucleotide sequence encoding an N-terminal SV40 NLS operably-linked to a site-directed endonuclease, such as a SpCas9 polypeptide, and a C-terminal nucleoplasmin NLS operably-linked to the site-directed endonuclease, such as the SpCas9 polypeptide. In some embodiments, the nucleoplasmin NLS comprises the amino acid sequence of SEQ ID NO: 7. In some embodiments, the SV40 NLS comprises the amino acid sequence of SEQ ID NO: 8. In some embodiments, the site-directed endonuclease comprises the amino acid sequence of SEQ ID NO: 6.

In some embodiments, the mRNA purified according to the methods disclosed herein comprises (i) a 5′ UTR; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR.

In some embodiments, the mRNA purified according to the methods disclosed herein comprises (i) a 5′ UTR; (ii) an open reading frame (ORF) comprising a nucleotide sequence that encodes a site-directed endonuclease, such as a SpCas9 polypeptide, wherein the nucleotide sequence is set forth by SEQ ID NO: 4; and (iii) a 3′ UTR.

In some embodiments, the 5′ UTR of any of the foregoing mRNA is a 5′ UTR described herein. In some embodiments, the 3′ UTR of any of the foregoing mRNA is a 3′ UTR described herein.

In some embodiments, the mRNA purified according to the methods disclosed herein comprises (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12.

In some embodiments, the mRNA purified according to the methods disclosed herein comprises (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12.

In some embodiments, the mRNA purified according to the methods disclosed herein comprises (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12.

In some embodiments, the mRNA purified according to the methods disclosed herein comprises (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12.

In some embodiments, any of the foregoing mRNA further comprises a poly-A tail, such as one described herein. In some embodiments, the poly-A tail comprises the nucleotide sequence of SEQ ID NO: 13.

In some embodiments, the mRNA purified according to the methods disclosed herein comprises a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 2. In some embodiments, the disclosure provides an mRNA comprising a nucleotide sequence that is 100% identical to the nucleotide sequence of SEQ ID NO: 2.

In some embodiments, the mRNA purified according to the methods disclosed herein comprises a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of ID NO: 14. In some embodiments, the disclosure provides an mRNA comprising a nucleotide sequence that is 100% identical to the nucleotide sequence of SEQ ID NO: 14.

In some embodiments, any of the foregoing mRNA comprise at least one chemically modified nucleoside. In some embodiments, the chemically modified nucleoside is selected from pseudouridine, N-1-methylpseudouridine, and 5-methoxyuridine. In some embodiments, the chemically modified nucleoside is N1-methylpseudouridine. In some embodiments, at least about 80% or more (e.g., about 85%, 90%, 91%, 92%, 93%, 94%, 95%, 96%, 97%, 98%, 99%) of uridines in the mRNA are modified or replaced with N1-methylpseudouridine. In some embodiments, 100% of the uridines in the mRNA are modified or replaced with N1-methylpseudouridine.

In some embodiments, the mRNA purified according to the methods disclosed herein comprises (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N-1-methylpseudouridine.

In some embodiments, the mRNA purified according to the methods disclosed herein comprises (i) a 5′ UTR, wherein the 5′ UTR comprises the nucleotide sequence of SEQ ID NO: 10; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N-1-methylpseudouridine.

In some embodiments, the mRNA purified according to the methods disclosed herein comprises (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N-1-methylpseudouridine.

In some embodiments, the mRNA purified according to the methods disclosed herein comprises (i) a 5′ UTR, wherein the 5′ UTR comprises the nucleotide sequence of SEQ ID NO: 15; (ii) an open reading frame (ORF) comprising a nucleotide sequence that encodes a site-directed endonuclease, such as a SpCas9 polypeptide, wherein the nucleotide sequence is set forth by the nucleotide sequence of SEQ ID NO: 4; and (iii) a 3′ UTR, wherein the 3′ UTR comprises the nucleotide sequence of SEQ ID NO: 12, wherein 100% of the uridines of the mRNA are modified or replaced with N-1-methylpseudouridine.

In some embodiments, the mRNA purified according to the methods disclosed herein comprises a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 2, wherein 100% of the uridines of the mRNA are modified or replaced with N-1-methylpseudouridine.

In some embodiments, the mRNA purified according to the methods disclosed herein comprises a nucleotide sequence that is 100% identical to the nucleotide sequence of SEQ ID NO: 2, wherein 100% of the uridines of the mRNA are modified or replaced with N-1-methylpseudouridine.

In some embodiments, the mRNA purified according to the methods disclosed herein comprises a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of ID NO: 14, wherein 100% of the uridines of the mRNA are modified or replaced with N-1-methylpseudouridine.

In some embodiments, the mRNA purified according to the methods disclosed herein comprises a nucleotide sequence that is 100% identical to the nucleotide sequence of SEQ ID NO: 14, wherein 100% of the uridines of the mRNA are modified or replaced with N-1-methylpseudouridine.

In some embodiments, any of the foregoing mRNA further comprises a 5′ cap, such as one described herein. In some embodiments, the 5′ cap is a cap-0, a cap-1, or a cap-2 structure.

Kits

The present disclosure provides kits for carrying out the methods described herein. In some embodiments, the kit comprises: i) a reactive moiety coupled to the monolithic matrix, and ii) a ligand that binds to an mRNA; and instructions for purifying a sample comprising an mRNA of interest using the monolithic matrix followed by adsorption chromatography to separate dsRNA from the mRNA of interest. In some embodiments, the kit comprises a substrate for adsorption chromatography, and instructions for removing dsRNA from a solution comprising an mRNA of interest, wherein the solution was previously purified with a monolithic matrix comprising a ligand comprising: i) a reactive moiety coupled to the monolithic matrix, and ii) a ligand that binds to the mRNA. In some embodiments, the kit comprises a monolithic matrix comprising a ligand comprising i) a reactive moiety coupled to the monolithic matrix, and ii) a ligand that binds to an mRNA; a substrate for adsorption chromatography; and instructions for purifying a sample comprising an mRNA of interest using the monolithic matrix followed by adsorption chromatography to separate dsRNA from the mRNA of interest. Components of a kit can be in separate containers, or combined in a single container.

Any kit described above can further comprise one or more additional reagents, where such additional reagents are selected from a buffer, a buffer for introducing a nucleic acid or delivery system described herein into a cell, a wash buffer, a control reagent, a control vector, a control RNA polynucleotide, a reagent for in vitro production of the polypeptide (e.g., SpCas9 polypeptide) from an mRNA described herein, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the SpCas9 polypeptide encoded by an mRNA described herein, or improve the specificity of targeting.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a,” “an,” and “the” include plural references unless the context clearly dictates otherwise. For example, the term “a cell” includes one or more cells, comprising mixtures thereof. “A and/or B” is used herein to include all of the following alternatives: “A,” “B,” “A or B,” and “A and B.”

The term “about,” as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

It is understood that aspects and embodiments of the disclosure described herein include “comprising,” “consisting,” and “consisting essentially of” aspects and embodiments.

The terms “individual,” “subject,” “host,” and “patient,” are used interchangeably herein and refer to any mammalian subject, such as human (e.g., human subjects), non-human mammals and non-human primates, for whom diagnosis, treatment, or therapy is desired, particularly humans.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein. In some embodiments, a nucleic acid molecule of the disclosure is an mRNA described herein, such as an mRNA encoding a site-directed endonuclease, such as a SpCas9 polypeptide described herein. In some embodiments, a nucleic acid molecule of the disclosure is a gRNA described herein. In some embodiments, a nucleic acid molecule of the disclosure is a donor polynucleotide described herein.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, or mafft.cbrc.jp/alignment/software/. See, e.g., Altschul et al. (1990), J. Mol. Biol. 215:403-10. Sequence alignments standard in the art are used according to the disclosure to determine nucleotides in an mRNA described herein that “correspond to” nucleotides in another mRNA. The nucleotides of the first mRNA that correspond to nucleotides of the second mRNA appear at the same position in alignments of the sequences.

A DNA sequence that “encodes” a particular RNA is a DNA nucleic acid sequence that is transcribed into RNA. A DNA polynucleotide can encode an RNA (mRNA) that is translated into protein, or a DNA polynucleotide can encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector (e.g., an AAV). As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

The term “operably linked,” as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements are contiguous or non-contiguous.

As used herein, the term “manipulating” or “editing” DNA encompasses binding, or cleaving (i.e., cutting) one or both strands of the DNA, or encompasses modifying the DNA or a polypeptide associated with the DNA. Manipulating or editing DNA can silence, activate, or modulate (either increase or decrease) the expression of an RNA or polypeptide encoded by the DNA.

As used herein, the terms “nuclease” and “endonuclease” are used interchangeably herein to mean an enzyme which possesses endonucleolytic catalytic activity for polynucleotide cleavage. The term includes site-specific endonucleases such as site-specific endonucleases of clustered, regularly interspaced, short palindromic repeat (CRISPR) systems such as, e.g., Cas polypeptides, e.g., a SpCas9 polypeptide.

By “site-directed endonuclease,” it is meant a polypeptide (e.g., Cas9 polypeptide, SpCas9 polypeptide) that binds gRNA and is targeted to a specific DNA sequence. A site-directed endonuclease as described herein is targeted to a specific DNA sequence by the RNA molecule (e.g., gRNA) to which it is bound. The RNA molecule comprises a sequence that binds, hybridizes to, or is complementary to a target sequence within the target DNA, thus targeting the bound polypeptide (e.g., Cas9 polypeptide, SpCas9 polypeptide) to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain aspects, a complex comprising a guide RNA and a site-directed modifying polypeptide is used for targeted double-stranded DNA cleavage.

As used herein, the term “SpCas9 polypeptide” refers to a Cas9 polypeptide derived from S. pyogenes. As used herein, the term “SpCas9 mRNA” refers to an mRNA encoding a SpCas9 polypeptide.

### Exemplary Embodiments

1. A method for purifying messenger RNA (mRNA) encoding a DNA endonuclease from a sample, the method comprising: (a) loading the sample comprising the mRNA onto a monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix after one or more contaminants have been separated from the bound mRNA; and (c) separating the mRNA from dsRNA by adsorption chromatography, thereby resulting in a purified mRNA solution.

2. A method for separating double stranded RNA (dsRNA) from mRNA encoding a DNA endonuclease, the method comprising: (a) loading a sample comprising the mRNA with monolithic matrix comprising a poly(dT) or poly(U) nucleic acid molecule linked/coupled to the monolithic matrix under conditions allowing the mRNA to hybridize with the poly(dT) or poly(U) nucleic acid molecule; (b) eluting the mRNA from the monolith matrix, thereby resulting in a semi-purified mRNA solution; and; (c) separating the mRNA in the semi-purified mRNA solution from dsRNA by adsorption chromatography, thereby resulting in a purified mRNA solution.

3. The method of embodiment 1 or embodiment 2, wherein the DNA endonuclease is SpCas9.

4. The method of any one of embodiments 1-3, wherein nucleotides in the mRNA are modified.

5. The method of embodiment 4, wherein the uridines in the mRNA are replaced with N-1-methylpseudouridine, pseudouridine, and/or 5-methoxyuridine.

6. The method of any one of embodiments 1-3, wherein the mRNA comprises the nucleotide sequence of SEQ ID NO: 2, and wherein uridines in the mRNA are replaced with N1-methylpseudouridine;

7. A method for purifying mRNA encoding SpCas9 from a sample, comprising: (a) loading the sample comprising the mRNA onto a monolithic matrix comprising a poly(dT) or poly(U) nucleic acid linked/coupled to the monolithic matrix such that the mRNA binds the column, wherein the mRNA comprises the nucleotide sequence of SEQ ID NO: 2, and wherein uridines in the mRNA are replaced with N-1-methylpseudouridine; (b) eluting the mRNA from the column after one or more contaminants have been separated from the bound mRNA; and (c) separating the mRNA of (b) from dsRNA by adsorption chromatography, thereby resulting in a purified mRNA solution.

8. The method of any one of embodiments 1-7, wherein the one or more contaminants are selected from the group of proteins, unreacted nucleotides, plasmid DNA, CAP analogues, partial transcripts, dsRNA side products and enzymes.

9. The method of any one of embodiments 1-8, wherein the mRNA comprises a poly(a) tail and wherein the one or more contaminants lack a poly(a) tail.

10. The method of any one of embodiments 1-9, wherein the mRNA is transcribed from a linearized DNA plasmid via an in vitro transcription (IVT) reaction.

11. The method of any one of embodiments 1-8, wherein 100% of the uridines in the mRNA are modified and/or replaced with N-1-methylpseudouridine.

12. A method for producing purified mRNA encoding a DNA endonuclease, comprising: (a) linearizing a codon optimized DNA plasmid encoding the endonuclease; (b) subjecting the plasmid of (a) to an IVT reaction in the presence of a modified uridine nucleotide to synthesize mRNA comprising the modified uridine nucleotide; (c) purifying the mRNA by a method comprising: (i) loading the sample comprising the mRNA onto a monolithic matrix comprising a poly(dT) or poly(U) nucleic acid linked/coupled to the monolithic matrix such that the mRNA binds the column, wherein the mRNA comprises the nucleotide sequence of SEQ ID NO: 2, and wherein uridines in the mRNA are replaced with N-1-methylpseudouridine; (ii) eluting the mRNA from the column after one or more contaminants have been separated from the bound mRNA; and (iii) separating the mRNA of (b) from dsRNA by adsorption chromatography, thereby resulting in an semi-purified mRNA solution; (iv) separating the mRNA in the semi-purified mRNA solution from dsRNA by adsorption chromatography, thereby producing a purified mRNA solution.

13. The method of any one of embodiments 1-12, wherein adsorption chromatography comprises a liquid mobile phase and a solid stationary support.

14. The method of embodiment 13, wherein the solid stationary support is non-polar.

15. The method of any one of embodiments 1-15, wherein adsorption chromatography is reverse phase chromatography.

16. The method of embodiment 15, where the sample is loaded onto the column for reverse phase chromatography and the elution buffer is about 35% to about 55% Buffer B, optionally about 50% Buffer B, and the remainder comprising Buffer A, wherein Buffer A comprises 0.1M TEAA and Buffer B comprises 0.1M TEAA and 25% acetonitrile

17. The method of embodiment 15 or 16, wherein the flow rate through the column is about 0.5 mL/min-5.0 mL/min, optionally about 3 mL/min.

18. The method of any one of embodiments 15-17, wherein the mRNA is loaded onto the column for reverse phase chromatography at a concentration of 0.05-5.00 mg/mL.

19. The method of any one of embodiments 1-18, wherein the purified mRNA solution has less than 0.015% dsRNA, or wherein dsRNA is not detectable in the purified mRNA solution.

20. The method of any one of embodiments 1-19, wherein the purified mRNA solution is further processed to exchange the buffer.

21. The method of embodiment 20, wherein the buffer is exchanged by a tangential flow filtration (TFF) system.

22. The method of any one of embodiments 1-21, wherein the purified mRNA or mRNA solution has reduced immunogenicity compared to mRNA purified via step (a) and not via adsorption chromatography.

23. The method of any one of embodiments 12-22, wherein the purified mRNA solution has reduced immunogenicity compared to mRNA solution purified via step (c)(i) without step (c)(ii).

24. The method of any one of embodiments 22-23, wherein reduced immunogenicity comprises a reduction in serum levels of MCP1, IL-6 or both.

25. The method of any one of embodiments 1-24, wherein the mRNA comprises a nucleotide sequence comprising the sequence set forth in SEQ ID NO: 2.

26. A composition of purified mRNA produced by the method of any one of embodiments 1-25.

27. A kit comprising: a monolithic matrix comprising a ligand comprising: i) a reactive moiety coupled to the monolithic matrix, and ii) a ligand that binds to an mRNA; a substrate for adsorption chromatography; and instructions for purifying a sample comprising an mRNA of interest using the monolithic matrix followed by adsorption chromatography to separate dsRNA from the mRNA of interest.

21. A kit comprising a monolithic matrix comprising a ligand comprising: i) a reactive moiety coupled to the monolithic matrix, and ii) a ligand that binds to an mRNA; and instructions for purifying a sample comprising an mRNA of interest using the monolithic matrix followed by adsorption chromatography to separate dsRNA from the mRNA of interest.

22. A kit comprising a substrate for adsorption chromatography, and instructions for removing dsRNA from a solution comprising an mRNA of interest, wherein the solution was previously purified with a monolithic matrix comprising a ligand comprising: i) a reactive moiety coupled to the monolithic matrix, and ii) a ligand that binds to the mRNA.

### EXAMPLES

The present disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

The practice of the present disclosure will employ, unless otherwise indicated, techniques of molecular biology, microbiology, cell biology, biochemistry, nucleic acid chemistry, and immunology, which are known to those skilled in the art. Such techniques are explained in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Beaucage et al. eds., Current Protocols in Nucleic Acid Chemistry, John Wiley & Sons, Inc., New York, 2000, (including supplements through 2014), Gene Transfer and Expression in Mammalian Cells (Makrides, ed., Elsevier Sciences B.V., Amsterdam, 2003); and Current Protocols in Immunology (Horgan K and S. Shaw (1994), including supplements through 2014). As appropriate, procedures involving the use of commercially available kits and reagents are generally carried out in accordance with manufacturer defined protocols and/or parameters unless otherwise noted.

### Example 1—Synthesis and Purification of SpCas9 mRNA

The following Example describes a protocol for the synthesis and purification (by adsorption chromatography) of SpCas9 mRNA that resulted in SpCas9 mRNA with reduced immunogenicity compared to SpCas9 mRNA purified by a means other than adsorption chromatography.

Restriction Digest: DNA Template Linearization

The DNA plasmid stock concentration was approximately 5 mg/mL, which was determined by diluting in water to an acceptable range for UV absorbance measurement by NanoDrop analysis (20-2000 μg/mL).

The volume of each component to add to the reaction mix was determined using the target concentration of each component, shown in Table 1. Component volumes were based on a starting DNA plasmid concentration of 5.0 mg/mL. The values were recalculated based on exact concentration of the plasmid DNA.

TABLE 1
Restriction Digest Reaction Component Mixture

| | | | | | | Component |
|---|---|---|---|---|---|---|
| No. | Component | Stock Conc. | | Target Conc. | | Volume (μL) |
| 1 | Water | N/A | | N/A | | 2622.9 |
| 2 | DNA Plasmid | 5.00 | μg/μL | 0.50 | μg/μL | 364.3 |
| 3 | CutSmart | 10X | | 1X | | 364.3 |
| | Buffer | | | | | |
| 4 | Restriction | 10.00 | U/μL | 0.80 | U/μL | 291.4 |
| | Enzyme BspQI | | | | | |

The reaction mixture identified in Table 1 was prepared in a 15 mL Falcon tube and gently mixed using a pipette. The mixture was evenly split into three 1.5 mL Eppendorf tubes for incubation. The tubes were incubated for 2.5 hours at 50° C. in a heated sonication water bath. Following incubation, the enzyme was deactivated by placing the reaction on a heating block at 80° C. for 20 min. The tubes were then placed on ice following enzyme deactivation.

Gel-based verification was used to confirm plasmid linearization. Briefly, a 1:10 dilution of 1 kb ladder is prepared, and 10 μl of 1 kb ladder is added to 90 μl water. Additionally, a 1:500 dilution of the DNA plasmid was prepared to obtain a final concentration of about 10 μg/mL. A 1:50 dilution of the linearized DNA template was prepared by adding 1 μL DNA template to 49 μL water for a final concentration of about 10 μg/mL. Approximately 20 μl of each sample is loaded onto a 2% Agarose gel. Gel electrophoresis was run for 10 minutes to allow separation and to confirm plasmid linearization of restriction digest reaction mixture. The linearized plasmid was filtered using a 0.2 μm membrane syringe filter. Subsequent in vitro transcription was performed the same day or the linearized template was stored at −20° C. overnight.

In Vitro Transcription and mRNA Synthesis

A 10× Transcription Buffer was freshly prepared using 500 mM Tris-HCl, pH 7.5+300 mM MgCl₂+100 mM DTT. The buffer was mixed and filtered with 0.2 μm syringe filter.

A 0.02 U/μL IPPase solution was prepared by adding 2 mL water to a 1 bottle of 1 mg IPPase. The solution was mixed gently on a mixing plate for 20 minutes. The solution was then filtered with a 0.2 μm syringe filter.

The reaction mix according to Table 2 was prepared in a 50 mL Falcon tube.

TABLE 2
Transcription Reaction Component Mixture

| | | | | | | Component |
|---|---|---|---|---|---|---|
| No. | Component | Stock Conc. | | Target Conc. | | Volume (mL) |
| 1 | Water | N/A | | N/A | | 13.95 |
| 2 | 10X TXN | 10.0 | mM | 1.0 | mM | 3.60 |
| | Buffer | | | | | |
| 3 | ATP | 100 | mM | 5.0 | mM | 1.80 |
| 4 | CTP | 100 | mM | 5.0 | mM | 1.80 |
| 5 | GTP | 100 | mM | 5.0 | mM | 1.80 |
| 6 | Me1PsUTP | 100 | mM | 5.0 | mM | 1.80 |
| 7 | CleanCap AG | 100 | mM | 5.0 | mM | 1.80 |
| 8 | Linearized | 0.50 | mg/mL | 0.05 | mg/mL | 3.60 |
| | DNA plasmid | | | | | |
| 9 | IPPase | 0.40 | U/μL | 0.02 | U/μL | 1.80 |
| 10 | RNase | 40.0 | U/μL | 0.50 | U/μL | 0.45 |
| | Inhibitor | | | | | |
| 11 | T7 RNA | 50.0 | U/μL | 5.00 | U/μL | 3.60 |
| | Polymerase | | | | | |

The reaction was gently mixed by pipetting or inverting. The reaction mixture was split into three 50 mL Falcon tubes (approximately 12 mL per tube). The reaction was incubated at 37° C. for 3 hours in a sonication water bath. Following incubation, the samples were cooled by placing on ice or storing at 4° C.

The mRNA concentration in the transcription reaction mix was quantified using a Quant-iT™ RNA Assay Kit. Briefly, a 1:500 dilution of the transcription reaction sample was prepared and mixed by pipette. The transcription reaction sample was further serially diluted 3×s in half for a total of 4 dilutions per sample. An mRNA standard was diluted to 10 μg/mL in 50 μl. The mRNA standard was further serially diluted 6×s in half for a total of 7 dilutions. A Quant-iT™ kit working solution was prepared from a 200× stock. 10 μl of each sample was added to a 96 well plate to read in duplicate, and 10 μl of each N=2 mRNA standard was added to the plate.

200 μl of the Quant-iT™ kit working solution was added to each well and mix with a pipette. A plate reader was used to measure the fluorescence at 644/674 nm. The mRNA concentration in each sample was quantified based on the mRNA standard ladder, with the final concentration being about 5.0 mg/mL.

DNA Template Removal Via DNase Reaction

The component volumes required to perform the DNase reaction were identified in Table 4 and are based on a Transcription reaction concentration of 5.00 mg/mL. The component volumes were recalculated based on the target component concentration and the transcription reaction concentration.

TABLE 4
DNase Reaction Component Mixture

| | | | | Total | Component |
|---|---|---|---|---|---|
| | | | | Component | Volume per |
| No. | Component | Stock Conc. | | Volume (mL) | Tube (mL) |
| 1 | Water | N/A | | 96.3 | 32.1 |
| 2 | 10X DNase | 10.0 | mg/mL | 15.0 | 5.0 |
| | Buffer | | | | |
| 3 | Transcription | 5.00 | mg/mL | 36.0 | 12.0 |
| | Reaction | | | | |
| 4 | DNase I | 2000 | U/mL | 2.7 | 0.9 |

A 10× DNase Buffer was prepared that includes 100 mM Tris-HCl, pH 7.5+25 mM MgCl₂+5 mM CaCl₂) pH 7.5. The buffer is filtered with a 0.2 μm syringe filter.

Components in Table 4 were added to the three 50 mL transcription reaction tubes and the tubes were mixed by inverting. The tubes were incubated at 37° C. for 45 minutes in a sonication water bath. Following incubation, the reaction was quenched by adding 1.440 mL of 0.5 M EDTA to each reaction tube. The reaction tubes were stored on ice or at 4° C. before gel verification and Quant-iT analysis.

Confirmation of template removal was performed using a 1% Agarose Gel. The 1:10 diluted ladder was prepared by adding 10 μl 1 kb ladder to 90 μl water. A 1:50 dilution of the DNase reaction was prepared. A 1:50 dilution of the transcription reaction dilution was prepared. Subsequently, 20 μl of each sample to the agarose gel. The E-gel reader was run for 10 minutes to allow separation and confirm template removal in the DNase reaction.

The mRNA concentration in the DNase reaction mix was quantified using the Quant-iT™ RNA Assay Kit. Briefly, a 1:100 dilution of the DNase reaction sample was prepared. The DNase reaction sample was further serially diluted 3×s in half for a total of 4 dilutions per sample. The mRNA standard described above was used for this analysis. A Quant-iT™ kit working solution was prepared from a 200× stock. 10 μl of each sample was added to the 96 well plate to read in duplicate, and 10 μl of each N=2 mRNA standard was added to Row A of the plate. Subsequently, 200 μl of the Quant-iT™ kit working solution was added to each well and mixed with pipette. A plate reader was used to measure the fluorescence at 644/674 nm. The mRNA concentration in each sample was quantified based on the mRNA standard ladder, with the final concentration being about 1.0 mg/mL.

Oligo-dT Purification of mRNA

Samples were prepared for Oligo-dT purification by diluting the mRNA to 0.2 mg/mL using equilibrium buffer, which is 50 mM Sodium Phosphate, 200 mM Sodium Sulfate, 10 mM EDTA, pH 7.0. The mRNA was filtered using a 0.2 μm vacuum filter. The mRNA was stored at 4° C. for short-term storage or −20° C. for long term storage.

Prior to purification, the column chromatography system was cleaned. Specifically, the column lines are cleaned with 0.5 N NaOH then rinsed with filtered Milli-Q water. Buffers in Table 5 were prepared for use in the oligo-dT purification.

TABLE 5
Oligo-dT Purification Method

| Column | |
|---|---|
| Line | Buffer |
| A1 | 50 mM Sodium Phosphate, 200 mM Sodium Sulfate, 10 mM |
| | EDTA, pH 7.0 |
| A2 | 50 mM Sodium Phosphate, 10 mM EDTA, pH 7.0 |
| B1 | 10 mM Tris |
| B2 | 0.1N NaOH |

The column chromatography system was then primed with each buffer, the S1 line was primed with equilibrium buffer, and the Oligo-dT column (CIMmultus Oligo dT18-8 Advanced Composite Column (Pores 2 μm)) was connected. The column was equilibrated in equilibrium buffer until the conductivity, etc. stabilized (5-10 mL/min for about 5-10 minutes). A 50 mL aliquot of filtered 0.2 mg/mL mRNA (20 mg mRNA) was added to a 50 mL Falcon tube and connected to the column chromatography system through the S1 line. The system was run using the mRNA Purification method detailed in Table 6 and fractions were collected from the column.

TABLE 6
RNA Purification Method for an 8 mL Column

| | | | | Flow | |
|---|---|---|---|---|---|
| | | | Column | Rate | Column |
| Step | Description | Buffer | Line | (mL/min) | Volumes |
| 1 | Clean | 0.1N NaOH | B2 | 8 | 5 |
| 2 | Equilibrate | Sodium Sulfate | A1 | 8 | 15 |
| 3 | Sample | Sample | S1 | 8 | 1 |
| | Application | | | | |
| 4 | Column Wash 1 | Sodium Sulfate | A1 | 8 | 12 |
| 5 | Column Wash 2 | Sodium Phosphate | A2 | 8 | 20 |
| 6 | mRNA Elution | 10 mM Tris | B1 | 8 | 12 |
| 7 | Clean | 0.1N NaOH | B2 | 8 | 5 |

The mRNA concentration in the collected mRNA fractions was quantified by NanoDrop. Purified mRNA is stored at 4° C.

Reverse Phase Purification of mRNA

The column chromatography system was cleaned as described above and primed with the buffers identified below in Table 7. Line S1 was primed with 10 mM Tris. The column (70.69 mL RiboSep RNA Column Semi Prep, 100×30 mm) was connected and equilibrated in Equilibrium buffer until the conductivity, etc. stabilized (3 mL/min for about 5-10 minutes).

TABLE 7
Reverse Phase Purification Method

| Column | | | Volume |
|---|---|---|---|
| Line | Buffer | Buffer | Required (L) |
| A1 | 0.1M TEAA, pH 7.0 | A | 5.5 |
| B1 | 0.1M TEAA, 25% acetonitrile, pH 7.0 | B | 7.0 |
| A2 | 75% Acetonitrile | D | 12.0 |
| B2 | 0.1N NaOH | Clean | 6.0 |

Next, 20-40 mg mRNA from the Oligo-dT purification fractions was combined into a 150 mL sterile bottle. The mRNA concentration are approximately 0.20-0.40 mg/mL and combined concentration was confirmed by UV absorbance measurement using NanoDrop. The mRNA sample was connected to the S1 line and maintained on ice during purification to maintain RNA stability.

The reverse phase purification was performed according to the method detailed in Table 8 below and fractions were collected from the column using a fraction collector.

TABLE 8
RNA Purification Method for an 70.69 mL Column

| | | | | Flow | |
|---|---|---|---|---|---|
| | | | Column | Rate | Column |
| Step | Description | Buffer | Line | (mL/min) | Volumes |
| 1 | Clean | 0.1N NaOH | B2 | 3 | 3 |
| 2 | Flush | 100% Buffer B | A1 | 3 | 3 |
| 3 | Equilibrate | 50% Buffer A, | A1/B1 | 3 | 3 |
| | | 50% Buffer B | | | |
| 4 | Sample | Sample | S1 | 3 | 1 |
| | Application | | | | |
| 5 | ssRNA Elution | 50% Buffer A, | A1/B1 | 3 | 8 |
| | | 50% Buffer B | | | |
| 6 | dsRNA Elution | 100% Buffer B | B1 | 3 | 3 |
| 7 | Clean | 0.1N NaOH | B2 | 3 | 4 |
| 8 | Storage | Buffer D | A2 | 3 | 5 |

Once the method was complete, fractions containing mRNA and dsRNA were separately collected. The dsRNA fractions were used for dsRNA detection in the mRNA sample. The purified mRNA was stored at 4° C.

mRNA Buffer Exchange and Concentration

A tangential flow filtration (TFF) system was used for buffer exchange. The TFF system was cleaned using 0.5 N NaOH for 1 hour then rinsed with filtered Milli-Q water until the pH neutralized. The TFF system was then equilibrated with 1 mM Sodium Citrate, pH 6.4 buffer.

The mRNA samples were prepared by combining up to 250 mL sample from reverse phase purification into sterile 250 mL centrifuge tubes. The mRNA sample was loaded through the inlet pump. The TFF system was set to Concentrate/Diafiltration (C/F) Mode with a concentration of 10×, 10 diavolumes, a flowrate of 90 mL/min, and a TMP pressure of 2 psi. Once the TFF run was complete, the sample was collected. The concentration of the collected mRNA was quantified by UV absorbance measurement using NanoDrop. The mRNA was stored at 4° C. until testing and analysis was complete. Long term storage was −80° C.

The dsRNA was also buffer exchanged and concentrated. A 100 K 15 mL Amicon Spin tubes was primed by rinsing with water. The dsRNA sample from Reverse Phase Purification was added to the primed Amicon Spin tubes. The sample was concentrated by 10× and buffer exchange was performed using 1 mM Sodium Citrate, pH 6.4 buffer. Four buffer exchange cycles were performed. The concentrated and buffer exchanged dsRNA was collected into 1.5 mL Eppendorf tubes. This dsRNA was used as a positive control for dsRNA ELISA testing of the mRNA sample.

The mRNA was filtered using sterile a 0.22 μm 50 mL vacuum filters in a biosafety cabinet. Analytical characterization of the final mRNA sample was performed. Briefly, full length purity analysis by Capillary Electrophoresis (CE). Endotoxin testing was performed using limulus amebocyte lysate (LAL) testing. Concentration was determined by A260/A280 Absorption measurement using NanoDrop. The dsRNA content was quantified by an ELISA-based assay. The mRNA was stored at −80° C. in 1 mM Sodium Citrate Buffer, pH 6.4.

### Example 2—Results

The protocol provided in Example 1 was followed and 44 mg of an SpCas9 mRNA (full length RNA set forth in SEQ ID NO: 2) was produced.

Sequences of mRNA encoding SpCas9

| | | | |
|---|---|---|---|
| Sequence | | | Amino Acid |
| Name | DNA SEQ ID NO: | RNA SEQ ID NO: | SEQ ID NO: |

Parent mRNA

| | | | |
|---|---|---|---|
| Full-length | 16 | 17 | — |
| mRNA | | | |

RNA-009 (Sequence Optimized from Parent mRNA)

| | | | |
|---|---|---|---|
| Full-length | 1 | 2 | — |
| mRNA | | | |
| 5′ UTR | 9 | 10 | — |
| Coding Region | 3 | 4 | 5 |
| 3′ UTR | 11 | 12 | — |
| Poly-A Tail | 13 | 13 | — |

The sequences Yield, capillary electrophoresis and dsRNA ELISA analysis was performed to evaluate the integrity of the mRNA produced.

Results showed that a 77% yield was observed after the reverse phase purification step. Distinct mRNA and dsRNA peaks were observed in each run demonstrating successful separation of the two nucleic components (data not shown).

Full length purity of the final mRNA product was assessed by capillary electrophoresis. Results indicated that the final mRNA product had a purity of 85% and a length of 4,138 nucleotides. The results presented as a narrow distribution with only a modest shoulder indicating majority full length product integrity.

A dsRNA ELISA was performed in order to quantitate the dsRNA in the mRNA produced by the protocol in Example 1. Results showed that the mRNA contained 0.69% dsRNA.

By analytical observation, the described protocol allowed for high integrity, low impurity Cas9 mRNA to be generated in high yield, as needed for downstream therapeutic application.

### Example 3—Comparison of Various mRNA Purification Methods

The following Example compared the final unmodified mRNA product from three transcription purification methods (RNeasy silica column, LiCl precipitation, and oligodT monolith purification), each followed by reverse-phase HPLC purification. The parameters investigated were full-length RNA purity (measured using capillary electrophoresis) and dsRNA removal (measured by dsRNA ELISA).

The full-length purity results are set forth in Tables 9 and 10.

TABLE 9
CE for mRNA after first purification step

| Purification | | |
|---|---|---|
| Method | Purity (%) | Length (nt) |
| RNeasy maxi kit | 81.3% | 4412 |
| LICl precipitation | 79.6% | 4387 |
| Oligo-DT | 82.0% | 4412 |

TABLE 10
CE for mRNA after RP-HPLC (after second purification step)

| Purification | | |
|---|---|---|
| Method | Purity (%) | Length (nt) |
| RNeasy maxi kit | 79.2% | 4404 |
| LICl precipitation | 74.8% | 4451 |
| Oligo-DT | 81.6% | 4414 |

As shown in Tables 9 and 10, all three methods yielded similar full-length RNA purity. Purification by oligodT and RNeasy yielded the highest full-length purity.

Results for the dsRNA ELISA are summarized in Table 11.

TABLE 11

| Purification | HPLC | Flow-through (FT) | 100% B peak |
|---|---|---|---|
| Method | input | peak (main peak) | (predicted dsRNA) |
| RNeasy | 0.80% | 1.15% | 14.5% |
| LiCl precipitation | 1.35% | <0.015% | 3.86% |
| Oligo-Dt | 0.72% | <0.15% | 5.87% |

As shown in Table 11, oligodT and LiCl precipitation both yielded dsRNA content below the level of detection of the assay. RNeasy purification yielded a product with 1.15% dsRNA, which was considered unacceptably high.

The concentration of remaining mRNA was determined by A260, A260/A280, A260/A230, the results of which as shown in Table 12.

TABLE 12
Concentration of remaining mRNA

| Purification | Conc. | | A260/ | A260/ | Volume | Mass |
|---|---|---|---|---|---|---|
| method | (μg/μl) | A260 | A280 | A230 | (μl) | (μg) |
| RNeasy | 2.125 | 53.12 | 2.15 | 2.27 | 65 | 138 |
| LiCl precipitation | 2.335 | 58.37 | 2.14 | 2.26 | 60 | 140 |
| Oligo-Dt | 2.045 | 21.14 | 2.12 | 2.25 | 75 | 153 |

Taking all data together, oligodT is either equivalent to or better than RNeasy and LiCl precipitation for purifying full length mRNA and removing dsRNA. Differences in full-length purity are small and likely insignificant between the three methods. RNeasy appears to be quite poor for dsRNA removal.

### Example 4—Identification of Optimized Conditions for Reverse Phase Chromatography

Oligo-dT purified mRNA, which was generated as described in Example 1, was used to determine the optimized buffer/flow and loading conditions for separating ssRNA from dsRNA with reverse phase chromatography. The Buffers A, B, C, and D from Example 1 were used with the purification method outlined in Table 8, except the ratio of Buffer A:Buffer B in step 3 was varied. The ratios of Buffer A: Buffer B was optimized to obtain purified ssRNA with little to no dsRNA. The amount of oligo-dT purified mRNA was also optimized as too much mRNA can result in flow through of both ssRNA and dsRNA, while too little can result in the binding of both ssRNA and dsRNA to the column.

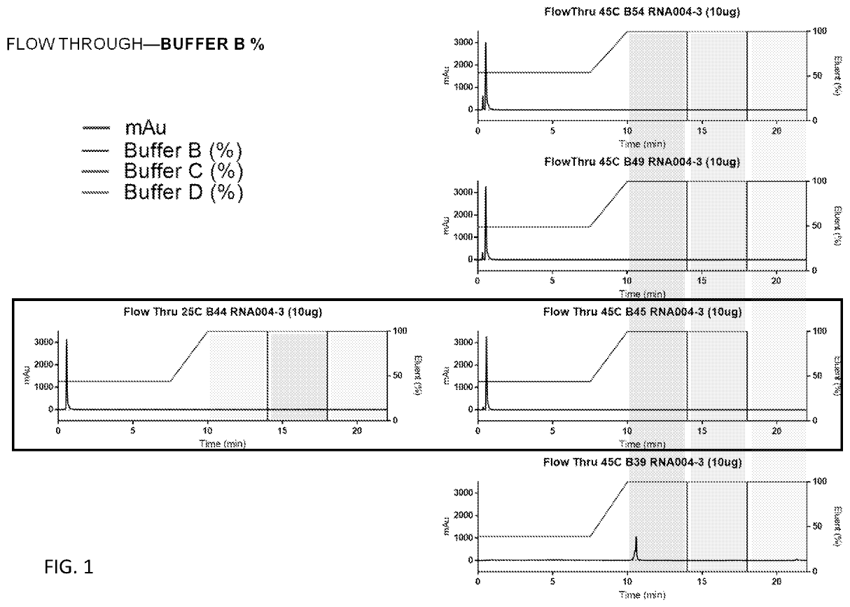

10 μg of oligo-dT purified mRNA was run through a column (Concise Separations RiboSep C18 PS-DVB 4.6×50 mm (RPC-99-3550); CV ˜0.83 ml), which was cleaned and primed, as essentially described in Example 1, with various Buffer B %'s (i.e., 54%, 49%, 45% of Buffer B at 45° C. and 44% of Buffer B at 25° C.) in steps 3 and 5. As shown in FIG. 1, ssRNA peak appeared about 1 min at a flow rate of 3 mL/min and where the Buffer B % was 54%, 49%, 45% at 45° C. and 44% at 25° C., whereas ssRNA appeared after 10 min when Buffer B % was 39%.

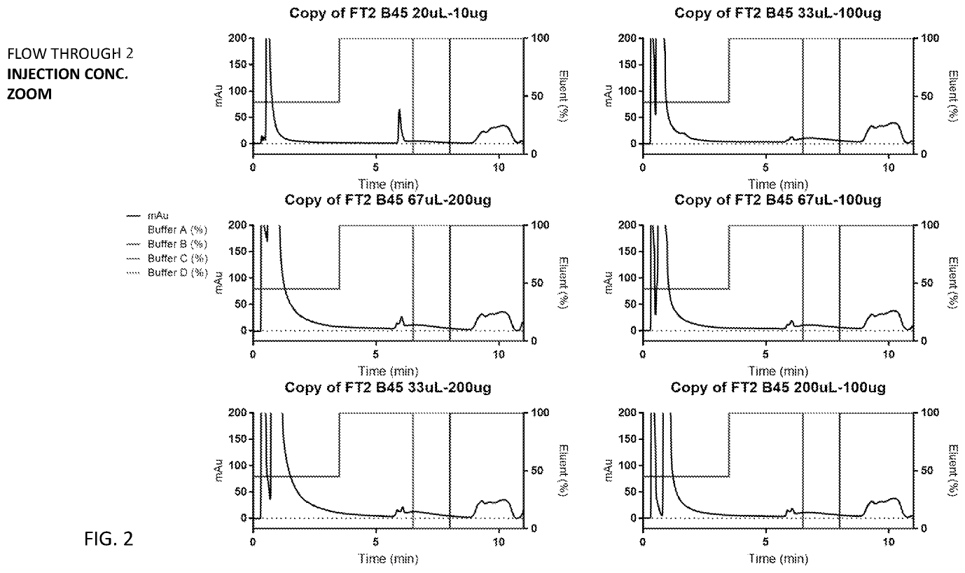

Various amounts of oligo-dT purified mRNA (100 μg/33 μL, 100 μg/67 μL, and 100 μg/200 μL, 0.33 μg/μL, 0.66 μg/μL and 0.5 μg/μL, respectively) were separately run through a column (Concise Separations RiboSep C18 PS-DVB 4.6×50 mm (RPC-99-3550); CV ˜0.83 ml) that was cleaned and primed, as generally described in Example 1, using 45% Buffer B. As shown in FIG. 2, these amounts were appropriate to get good flow through of ssRNA and separation from dsRNA.

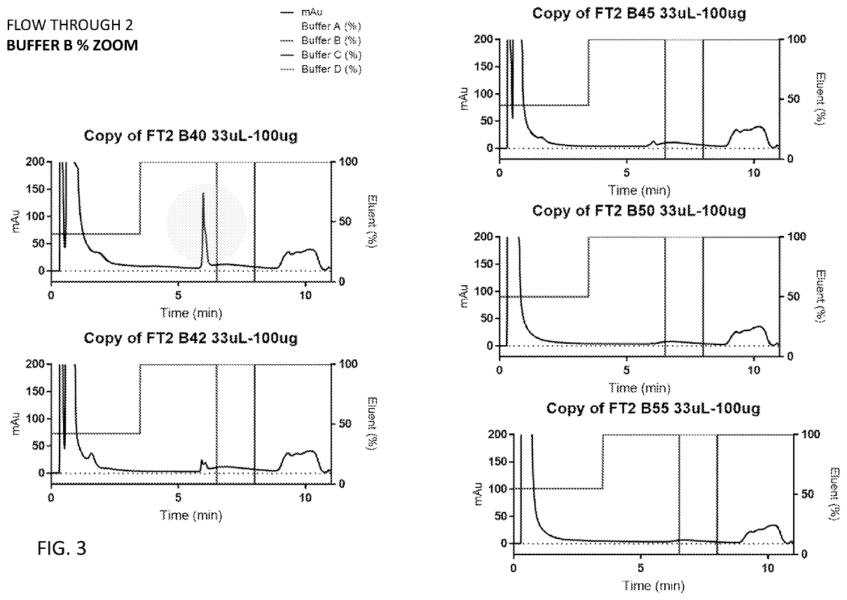

100 μg/33 μL (3.03 μg/μL) of oligo-dT purified mRNA was tested with various ratios of Buffer B. FIG. 3 shows that Buffer B at 40% had good flow through of ssRNA while the dsRNA peak eluted at ˜6 min.

1. A method for purifying messenger RNA (mRNA) encoding SpCas9 from a sample, the method comprising:
(a) loading the sample comprising the mRNA onto a monolithic matrix comprising a poly(dT) or poly(U) nucleic acid molecule linked/coupled to the monolithic matrix under conditions allowing the mRNA to hybridize with the poly(dT) or poly(U) nucleic acid molecule;
(b) eluting the mRNA from the monolith matrix after one or more contaminants have been separated from the bound mRNA; and
(c) separating the mRNA from dsRNA by adsorption chromatography, thereby resulting in a purified mRNA solution.

2. A method for separating double stranded RNA (dsRNA) from mRNA encoding SpCas9, the method comprising:
(a) loading a sample comprising the mRNA with monolithic matrix comprising a poly(dT) or poly(U) nucleic acid molecule linked/coupled to the monolithic matrix under conditions allowing the mRNA to hybridize with the poly(dT) or poly(U) nucleic acid molecule;
(b) eluting the mRNA from the monolith matrix, thereby resulting in a semi-purified mRNA solution; and;
(c) separating the mRNA in the semi-purified mRNA solution from dsRNA by adsorption chromatography, thereby resulting in a purified mRNA solution.

3. (canceled)

4. The method of claim 1, wherein nucleotides in the mRNA are modified.

5. The method of claim 4, wherein the uridines in the mRNA are replaced with N-1-methylpseudouridine, pseudouridine, and/or 5-methoxyuridine.

6. The method of claim 1, wherein the mRNA comprises the nucleotide sequence of SEQ ID NO: 2, and wherein uridines in the mRNA are replaced with N1-methylpseudouridine;

7. (canceled)

8. The method of claim 1, wherein the one or more contaminants are selected from the group of proteins, unreacted nucleotides, plasmid DNA, CAP analogues, partial transcripts, dsRNA side products and enzymes.

9. The method of claim 1, wherein the mRNA comprises a poly(a) tail and wherein the one or more contaminants lack a poly(a) tail.

10. The method of claim 1, wherein the mRNA is transcribed from a linearized DNA plasmid via an in vitro transcription (IVT) reaction.

11. The method of claim 1, wherein 100% of the uridines in the mRNA are modified and/or replaced with N-1-methylpseudouridine.

12. A method for producing purified mRNA encoding SpCas9, comprising:
(a) linearizing a codon optimized DNA plasmid encoding the endonuclease;
(b) subjecting the plasmid of (a) to an IVT reaction in the presence of a modified uridine nucleotide to synthesize mRNA comprising the modified uridine nucleotide;
(c) purifying the mRNA by a method comprising:
(i) loading the sample comprising the mRNA onto a monolithic matrix comprising a poly(dT) or poly(U) nucleic acid linked/coupled to the monolithic matrix such that the mRNA binds the column, wherein the mRNA comprises the nucleotide sequence of SEQ ID NO: 2, and wherein uridines in the mRNA are replaced with N-1-methylpseudouridine;
(ii) eluting the mRNA from the column after one or more contaminants have been separated from the bound mRNA; and
(iii) separating the mRNA of (b) from dsRNA by adsorption chromatography, thereby resulting in an semi-purified mRNA solution;
(iv) separating the mRNA in the semi-purified mRNA solution from dsRNA by adsorption chromatography,
thereby producing a purified mRNA solution.

13-14. (canceled)

15. The method of claim 1, wherein adsorption chromatography is reverse phase chromatography.

16. The method of claim 15, where the sample is loaded onto the column for reverse phase chromatography and an elution buffer is about 35% to about 55% Buffer B, optionally about 50% Buffer B, and the remainder comprising Buffer A, wherein Buffer A comprises 0.1 M TEAA and Buffer B comprises 0.1 M TEAA and 25% acetonitrile.

17. The method of claim 15, wherein the flow rate through the column is about 0.5 mL/min-5.0 mL/min, optionally about 3 mL/min.

18. The method of claim 15, wherein the mRNA is loaded onto the column for reverse phase chromatography at a concentration of 0.05-5.00 mg/mL.

19. The method of claim 1, wherein the purified mRNA solution has less than 0.015% dsRNA, or wherein dsRNA is not detectable in the purified mRNA solution.

20. The method of claim 1, wherein the purified mRNA solution is further processed to exchange a buffer.

21. The method of claim 20, wherein the buffer is exchanged by a tangential flow filtration (TFF) system.

22. The method of claim 1, wherein the purified mRNA or mRNA solution has reduced immunogenicity compared to mRNA purified via step (a) and not via adsorption chromatography.

23. The method of claim 12, wherein the purified mRNA solution has reduced immunogenicity compared to mRNA solution purified via step (c)(i) without step (c)(ii).

24-25. (canceled)

26. A composition of purified mRNA produced by the method of claim 1.

27-29. (canceled)


SEQUENCE LISTING (17 sequences)

SEQ ID NO: 1 - DNA, 4506 bp, Artificial Sequence
  Synthetic
agaggaaataagagagaaaagaagagtaagaagaaatataagagccaccatggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 4506 nt, Artificial Sequence
  Synthetic
agaggaaauaagagagaaaagaagaguaagaagaaauauaagagccaccauggccccuaagaagaagagaaaagucggaauucacggaguccccgccgccgacaaaaaguacuccauuggccuugauauuggaaccaacuccguggguugggccgugaucacugacgaguacaaggugccguccaagaaguucaaggugcuggggaacacugaccggcacucaauuaagaagaaccugauuggggcgcugcuguucgacuccggagaaaccgcggaggcuacccgccugaagcggacugcccggcggagauacacgcgcaggaagaaccggauuugcuaccuccaagaaaucuucagcaacgaaauggcaaagguggacgauuccuucuuccaucgccuggaagagagcuuccugguggaagaggacaagaagcacgaaagacacccgauuuucggcaacaucguggaugaggucgcauaccacgaaaaguaccccaccaucuaucaucuucggaagaagcuggucgacuccaccgauaaggccgaucugcgccugaucuacuuggcgcuggcucacaugauuaaguucagaggacacuuucugauagagggcgaccucaaucccgauaacuccgacguggauaagcuguucauccaacuggugcagacguacaaccaacuguuugaagagaauccaaucaacgccagcgggguggacgccaaggccauccuguccgcccggcugucaaaguccagacgccuggagaaucucaucgcgcaacucccuggcgaaaaaaagaacggacucuucgggaaucugauugcucugucccuggggcucacuccgaacuucaagucgaacuucgaccuggcggaggacgcuaagcugcagcuguccaaggacaccuacgaugacgaucuggauaaccuucuggcccagaucggggaucaauacgccgaucucuuccuggccgcaaagaacuugucggaugcuauucugcugagcgacauucugcgggucaauacugaaaucaccaaggcgccccugucggccagcaugaucaagcgcuacgacgaacaccaccaagaccugacucugcugaaggcccucgugcgccagcagcugccugaaaaguacaaggagauuuucuucgaccaguccaagaacggauacgccggauacauugacggaggggccagccaggaggaauuuuacaaauucaucaagcccauucucgagaaaauggacggaaccgaagaguugcucgugaagcugaacagagaggaucuccuccggaagcagcggaccuucgacaacgguuccaucccgcaccaaauccaccugggcgaauugcacgccauccuccggcggcaggaagauuucuacccauucuugaaggacaaucgcgaaaagaucgaaaagaucuugacuuuccgcaucccguacuacgugggcccucuggcccgcggcaacucccgcuucgcuuggaugacacggaaguccgaggaaaccauuacgcccuggaacuucgaggaagugguggacaagggggcguccgcccagagcuucaucgaacgcaugaccaauuucgacaagaaccucccgaacgaaaaagugcugccaaagcacucgcuccucuacgaauacuucaccguguacaacgagcugacuaaggucaaauacgugacugagggaaugcggaagccggccuuccugucgggagagcagaagaaggccauaguggacuugcuuuucaagacuaaccggaaggucacugugaagcaacucaaggaggacuacuucaagaagaucgaguguuucgacucgguggagaucucgggugucgaggaccgcuucaacgccucccugggaacuuaccacgaucugcugaagaucaucaaggacaaggacuuccucgauaacgaagaaaaugaggacauccucgaggauaucgugcugacccugaccuuguucgaggauagggagaugaucgaggagcggcucaagaccuacgcccaccuguuugacgacaaagugaugaagcaacugaaacggcggagguauaccggcuggggucggcugucccgcaagcugaucaacgggaucagggacaagcaguccggaaagaccauccucgacuuccuuaaguccgacggauucgcgaaccgcaacuucaugcaacuuauccacgacgacucgcugacauucaaggaagauauccagaaggcccagguguccggacagggggacucgcuucaugagcacaucgcuaaccuggccggaucccccgccauaaaaaagggcauucugcagaccgucaaagugguggaugagcuggucaaggucaugggccggcauaagccggaaaacaucgucaucgagauggcccgcgagaaccagacuacgcagaagggccagaagaacucccgggagcggaugaagcggauugaagagggcaucaaggagcucggcagccagauucugaaggaacaucccguggaaaacacccagcugcaaaacgaaaagcucuauuuguacuaucugcaaaacggacgcgauauguacguggaucaggagcuggacauuaacagacugagcgacuaugacguggaucacauugugccucaaagcuuccucaaggacgacucaauugacaacaagguccugaccagaagcgacaagaacagaggaaagucggauaaugugccguccgaagaaguggucaagaagaugaagaauuacuggagacagcuccugaaugcgaagcucauuacccagcggaaguucgauaaccugaccaaggccgaaagggguggacuguccgaacucgacaaagcuggcuucaucaagcgccaacuggucgaaaccaggcagaucaccaagcacgucgcccagauucuggacagccgcaugaacacuaaguacgacgagaacgauaagcugauccgcgaagugaaggucaucacccugaaguccaagcucguguccgacuuucggaaggauuuccaguuuuacaagguccgcgagaucaacaacuaccaucacgcccacgacgcguaccuuaacgcagucgugggaacggcucuuaucaagaaguacccaaagcuggagucggaauuuguguacggagacuacaaaguguacgacgugcgcaagaugaucgccaaaucugagcaagagaucgggaaggcaaccgccaaauacuucuucuacucaaacauuaugaauuuuuucaaaacugagauuacccuggcuaacggagaaauucggaagcgcccccugauugaaaccaacggagaaacuggagaaauugugugggacaagggacgggacuucgccaccguccgcaagguccucucaaugccccaagucaacaucgugaaaaagaccgaagugcaaaccggcggcuucucaaaggaguccauccugccuaagcgcaacagcgacaagcugauugccaggaagaaggacugggacccgaagaaguacggaggauuugauuccccuaccguggccuacuccgugcucgugguggccaaaguggaaaaggggaaauccaagaagcugaagucggugaaggagcuuuuggguaucaccaucauggaacgcuccucguucgaaaagaacccaaucgauuuccuggaagcuaaggguuauaaggaagugaaaaaggaccugauuaucaagcugcccaaguacucacuguucgagcuggaaaacggucggaaaaggaugcuggccagcgccggagaacuccagaagggaaacgaacuggcacugccguccaaauacgucaacuuccucuaccuugcaucccauuacgaaaaacucaagggaucgccggaggacaacgagcagaagcagcuuuucguggagcaacacaagcauuacuuggacgagaucaucgagcagauuuccgaguucucaaagcgcgugauccuggccgacgcaaaucuggacaagguccuguccgcguacaauaagcaucgggacaagccuauccgcgaacaggccgagaacaucauccaucuguucacucugacaaaccugggcgcacccgccgcguucaaguacuuugacaccaccaucgauaggaagcgauacaccucaacuaaggaaguguuggacgcgacccuuauccaucagucgaucaccgggcuguacgaaacacggaucgaccucagccaguugggaggcgacaagcgcccugcggcuaccaagaaggccggacaggccaagaagaagaaaugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 4203 bp, Artificial Sequence
  Synthetic
atggcccctaagaagaagagaaaagtcggaattcacggagtccccgccgccgacaaaaagtactccattggccttgatattggaaccaactccgtgggttgggccgtgatcactgacgagtacaaggtgccgtccaagaagttcaaggtgctggggaacactgaccggcactcaattaagaagaacctgattggggcgctgctgttcgactccggagaaaccgcggaggctacccgcctgaagcggactgcccggcggagatacacgcgcaggaagaaccggatttgctacctccaagaaatcttcagcaacgaaatggcaaaggtggacgattccttcttccatcgcctggaagagagcttcctggtggaagaggacaagaagcacgaaagacacccgattttcggcaacatcgtggatgaggtcgcataccacgaaaagtaccccaccatctatcatcttcggaagaagctggtcgactccaccgataaggccgatctgcgcctgatctacttggcgctggctcacatgattaagttcagaggacactttctgatagagggcgacctcaatcccgataactccgacgtggataagctgttcatccaactggtgcagacgtacaaccaactgtttgaagagaatccaatcaacgccagcggggtggacgccaaggccatcctgtccgcccggctgtcaaagtccagacgcctggagaatctcatcgcgcaactccctggcgaaaaaaagaacggactcttcgggaatctgattgctctgtccctggggctcactccgaacttcaagtcgaacttcgacctggcggaggacgctaagctgcagctgtccaaggacacctacgatgacgatctggataaccttctggcccagatcggggatcaatacgccgatctcttcctggccgcaaagaacttgtcggatgctattctgctgagcgacattctgcgggtcaatactgaaatcaccaaggcgcccctgtcggccagcatgatcaagcgctacgacgaacaccaccaagacctgactctgctgaaggccctcgtgcgccagcagctgcctgaaaagtacaaggagattttcttcgaccagtccaagaacggatacgccggatacattgacggaggggccagccaggaggaattttacaaattcatcaagcccattctcgagaaaatggacggaaccgaagagttgctcgtgaagctgaacagagaggatctcctccggaagcagcggaccttcgacaacggttccatcccgcaccaaatccacctgggcgaattgcacgccatcctccggcggcaggaagatttctacccattcttgaaggacaatcgcgaaaagatcgaaaagatcttgactttccgcatcccgtactacgtgggccctctggcccgcggcaactcccgcttcgcttggatgacacggaagtccgaggaaaccattacgccctggaacttcgaggaagtggtggacaagggggcgtccgcccagagcttcatcgaacgcatgaccaatttcgacaagaacctcccgaacgaaaaagtgctgccaaagcactcgctcctctacgaatacttcaccgtgtacaacgagctgactaaggtcaaatacgtgactgagggaatgcggaagccggccttcctgtcgggagagcagaagaaggccatagtggacttgcttttcaagactaaccggaaggtcactgtgaagcaactcaaggaggactacttcaagaagatcgagtgtttcgactcggtggagatctcgggtgtcgaggaccgcttcaacgcctccctgggaacttaccacgatctgctgaagatcatcaaggacaaggacttcctcgataacgaagaaaatgaggacatcctcgaggatatcgtgctgaccctgaccttgttcgaggatagggagatgatcgaggagcggctcaagacctacgcccacctgtttgacgacaaagtgatgaagcaactgaaacggcggaggtataccggctggggtcggctgtcccgcaagctgatcaacgggatcagggacaagcagtccggaaagaccatcctcgacttccttaagtccgacggattcgcgaaccgcaacttcatgcaacttatccacgacgactcgctgacattcaaggaagatatccagaaggcccaggtgtccggacagggggactcgcttcatgagcacatcgctaacctggccggatcccccgccataaaaaagggcattctgcagaccgtcaaagtggtggatgagctggtcaaggtcatgggccggcataagccggaaaacatcgtcatcgagatggcccgcgagaaccagactacgcagaagggccagaagaactcccgggagcggatgaagcggattgaagagggcatcaaggagctcggcagccagattctgaaggaacatcccgtggaaaacacccagctgcaaaacgaaaagctctatttgtactatctgcaaaacggacgcgatatgtacgtggatcaggagctggacattaacagactgagcgactatgacgtggatcacattgtgcctcaaagcttcctcaaggacgactcaattgacaacaaggtcctgaccagaagcgacaagaacagaggaaagtcggataatgtgccgtccgaagaagtggtcaagaagatgaagaattactggagacagctcctgaatgcgaagctcattacccagcggaagttcgataacctgaccaaggccgaaaggggtggactgtccgaactcgacaaagctggcttcatcaagcgccaactggtcgaaaccaggcagatcaccaagcacgtcgcccagattctggacagccgcatgaacactaagtacgacgagaacgataagctgatccgcgaagtgaaggtcatcaccctgaagtccaagctcgtgtccgactttcggaaggatttccagttttacaaggtccgcgagatcaacaactaccatcacgcccacgacgcgtaccttaacgcagtcgtgggaacggctcttatcaagaagtacccaaagctggagtcggaatttgtgtacggagactacaaagtgtacgacgtgcgcaagatgatcgccaaatctgagcaagagatcgggaaggcaaccgccaaatacttcttctactcaaacattatgaattttttcaaaactgagattaccctggctaacggagaaattcggaagcgccccctgattgaaaccaacggagaaactggagaaattgtgtgggacaagggacgggacttcgccaccgtccgcaaggtcctctcaatgccccaagtcaacatcgtgaaaaagaccgaagtgcaaaccggcggcttctcaaaggagtccatcctgcctaagcgcaacagcgacaagctgattgccaggaagaaggactgggacccgaagaagtacggaggatttgattcccctaccgtggcctactccgtgctcgtggtggccaaagtggaaaaggggaaatccaagaagctgaagtcggtgaaggagcttttgggtatcaccatcatggaacgctcctcgttcgaaaagaacccaatcgatttcctggaagctaagggttataaggaagtgaaaaaggacctgattatcaagctgcccaagtactcactgttcgagctggaaaacggtcggaaaaggatgctggccagcgccggagaactccagaagggaaacgaactggcactgccgtccaaatacgtcaacttcctctaccttgcatcccattacgaaaaactcaagggatcgccggaggacaacgagcagaagcagcttttcgtggagcaacacaagcattacttggacgagatcatcgagcagatttccgagttctcaaagcgcgtgatcctggccgacgcaaatctggacaaggtcctgtccgcgtacaataagcatcgggacaagcctatccgcgaacaggccgagaacatcatccatctgttcactctgacaaacctgggcgcacccgccgcgttcaagtactttgacaccaccatcgataggaagcgatacacctcaactaaggaagtgttggacgcgacccttatccatcagtcgatcaccgggctgtacgaaacacggatcgacctcagccagttgggaggcgacaagcgccctgcggctaccaagaaggccggacaggccaagaagaagaaatga

SEQ ID NO: 4 - RNA, 4203 nt, Artificial Sequence
  Synthetic
auggccccuaagaagaagagaaaagucggaauucacggaguccccgccgccgacaaaaaguacuccauuggccuugauauuggaaccaacuccguggguugggccgugaucacugacgaguacaaggugccguccaagaaguucaaggugcuggggaacacugaccggcacucaauuaagaagaaccugauuggggcgcugcuguucgacuccggagaaaccgcggaggcuacccgccugaagcggacugcccggcggagauacacgcgcaggaagaaccggauuugcuaccuccaagaaaucuucagcaacgaaauggcaaagguggacgauuccuucuuccaucgccuggaagagagcuuccugguggaagaggacaagaagcacgaaagacacccgauuuucggcaacaucguggaugaggucgcauaccacgaaaaguaccccaccaucuaucaucuucggaagaagcuggucgacuccaccgauaaggccgaucugcgccugaucuacuuggcgcuggcucacaugauuaaguucagaggacacuuucugauagagggcgaccucaaucccgauaacuccgacguggauaagcuguucauccaacuggugcagacguacaaccaacuguuugaagagaauccaaucaacgccagcgggguggacgccaaggccauccuguccgcccggcugucaaaguccagacgccuggagaaucucaucgcgcaacucccuggcgaaaaaaagaacggacucuucgggaaucugauugcucugucccuggggcucacuccgaacuucaagucgaacuucgaccuggcggaggacgcuaagcugcagcuguccaaggacaccuacgaugacgaucuggauaaccuucuggcccagaucggggaucaauacgccgaucucuuccuggccgcaaagaacuugucggaugcuauucugcugagcgacauucugcgggucaauacugaaaucaccaaggcgccccugucggccagcaugaucaagcgcuacgacgaacaccaccaagaccugacucugcugaaggcccucgugcgccagcagcugccugaaaaguacaaggagauuuucuucgaccaguccaagaacggauacgccggauacauugacggaggggccagccaggaggaauuuuacaaauucaucaagcccauucucgagaaaauggacggaaccgaagaguugcucgugaagcugaacagagaggaucuccuccggaagcagcggaccuucgacaacgguuccaucccgcaccaaauccaccugggcgaauugcacgccauccuccggcggcaggaagauuucuacccauucuugaaggacaaucgcgaaaagaucgaaaagaucuugacuuuccgcaucccguacuacgugggcccucuggcccgcggcaacucccgcuucgcuuggaugacacggaaguccgaggaaaccauuacgcccuggaacuucgaggaagugguggacaagggggcguccgcccagagcuucaucgaacgcaugaccaauuucgacaagaaccucccgaacgaaaaagugcugccaaagcacucgcuccucuacgaauacuucaccguguacaacgagcugacuaaggucaaauacgugacugagggaaugcggaagccggccuuccugucgggagagcagaagaaggccauaguggacuugcuuuucaagacuaaccggaaggucacugugaagcaacucaaggaggacuacuucaagaagaucgaguguuucgacucgguggagaucucgggugucgaggaccgcuucaacgccucccugggaacuuaccacgaucugcugaagaucaucaaggacaaggacuuccucgauaacgaagaaaaugaggacauccucgaggauaucgugcugacccugaccuuguucgaggauagggagaugaucgaggagcggcucaagaccuacgcccaccuguuugacgacaaagugaugaagcaacugaaacggcggagguauaccggcuggggucggcugucccgcaagcugaucaacgggaucagggacaagcaguccggaaagaccauccucgacuuccuuaaguccgacggauucgcgaaccgcaacuucaugcaacuuauccacgacgacucgcugacauucaaggaagauauccagaaggcccagguguccggacagggggacucgcuucaugagcacaucgcuaaccuggccggaucccccgccauaaaaaagggcauucugcagaccgucaaagugguggaugagcuggucaaggucaugggccggcauaagccggaaaacaucgucaucgagauggcccgcgagaaccagacuacgcagaagggccagaagaacucccgggagcggaugaagcggauugaagagggcaucaaggagcucggcagccagauucugaaggaacaucccguggaaaacacccagcugcaaaacgaaaagcucuauuuguacuaucugcaaaacggacgcgauauguacguggaucaggagcuggacauuaacagacugagcgacuaugacguggaucacauugugccucaaagcuuccucaaggacgacucaauugacaacaagguccugaccagaagcgacaagaacagaggaaagucggauaaugugccguccgaagaaguggucaagaagaugaagaauuacuggagacagcuccugaaugcgaagcucauuacccagcggaaguucgauaaccugaccaaggccgaaagggguggacuguccgaacucgacaaagcuggcuucaucaagcgccaacuggucgaaaccaggcagaucaccaagcacgucgcccagauucuggacagccgcaugaacacuaaguacgacgagaacgauaagcugauccgcgaagugaaggucaucacccugaaguccaagcucguguccgacuuucggaaggauuuccaguuuuacaagguccgcgagaucaacaacuaccaucacgcccacgacgcguaccuuaacgcagucgugggaacggcucuuaucaagaaguacccaaagcuggagucggaauuuguguacggagacuacaaaguguacgacgugcgcaagaugaucgccaaaucugagcaagagaucgggaaggcaaccgccaaauacuucuucuacucaaacauuaugaauuuuuucaaaacugagauuacccuggcuaacggagaaauucggaagcgcccccugauugaaaccaacggagaaacuggagaaauugugugggacaagggacgggacuucgccaccguccgcaagguccucucaaugccccaagucaacaucgugaaaaagaccgaagugcaaaccggcggcuucucaaaggaguccauccugccuaagcgcaacagcgacaagcugauugccaggaagaaggacugggacccgaagaaguacggaggauuugauuccccuaccguggccuacuccgugcucgugguggccaaaguggaaaaggggaaauccaagaagcugaagucggugaaggagcuuuuggguaucaccaucauggaacgcuccucguucgaaaagaacccaaucgauuuccuggaagcuaaggguuauaaggaagugaaaaaggaccugauuaucaagcugcccaaguacucacuguucgagcuggaaaacggucggaaaaggaugcuggccagcgccggagaacuccagaagggaaacgaacuggcacugccguccaaauacgucaacuuccucuaccuugcaucccauuacgaaaaacucaagggaucgccggaggacaacgagcagaagcagcuuuucguggagcaacacaagcauuacuuggacgagaucaucgagcagauuuccgaguucucaaagcgcgugauccuggccgacgcaaaucuggacaagguccuguccgcguacaauaagcaucgggacaagccuauccgcgaacaggccgagaacaucauccaucuguucacucugacaaaccugggcgcacccgccgcguucaaguacuuugacaccaccaucgauaggaagcgauacaccucaacuaaggaaguguuggacgcgacccuuauccaucagucgaucaccgggcuguacgaaacacggaucgaccucagccaguugggaggcgacaagcgcccugcggcuaccaagaaggccggacaggccaagaagaagaaauga

SEQ ID NO: 5 - protein, 1400 aa, Artificial Sequence
  Synthetic
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 6 - protein, 1367 aa, Artificial Sequence
  Synthetic
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 16 aa, Artificial Sequence
  Synthetic
KRPAATKKAGQAKKKK

SEQ ID NO: 8 - protein, 7 aa, Artificial Sequence
  Synthetic
PKKKRKV

SEQ ID NO: 9 - DNA, 49 bp, Artificial Sequence
  Synthetic
agaggaaataagagagaaaagaagagtaagaagaaatataagagccacc

SEQ ID NO: 10 - RNA, 49 nt, Artificial Sequence
  Synthetic
agaggaaauaagagagaaaagaagaguaagaagaaauauaagagccacc

SEQ ID NO: 11 - DNA, 114 bp, Artificial Sequence
  Synthetic
gcggccgcttaattaagctgccttctgcggggcttgccttctggccatgcccttcttctctcccttgcacctgtacctcttggtctttgaataaagcctgagtaggaagtctag

SEQ ID NO: 12 - RNA, 114 nt, Artificial Sequence
  Synthetic
gcggccgcuuaauuaagcugccuucugcggggcuugccuucuggccaugcccuucuucucucccuugcaccuguaccucuuggucuuugaauaaagccugaguaggaagucuag

SEQ ID NO: 13 - RNA, 140 nt, Artificial Sequence
  Synthetic
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - RNA, 4504 nt, Artificial Sequence
  Synthetic
aggaaauaagagagaaaagaagaguaagaagaaauauaagagccaccauggccccuaagaagaagagaaaagucggaauucacggaguccccgccgccgacaaaaaguacuccauuggccuugauauuggaaccaacuccguggguugggccgugaucacugacgaguacaaggugccguccaagaaguucaaggugcuggggaacacugaccggcacucaauuaagaagaaccugauuggggcgcugcuguucgacuccggagaaaccgcggaggcuacccgccugaagcggacugcccggcggagauacacgcgcaggaagaaccggauuugcuaccuccaagaaaucuucagcaacgaaauggcaaagguggacgauuccuucuuccaucgccuggaagagagcuuccugguggaagaggacaagaagcacgaaagacacccgauuuucggcaacaucguggaugaggucgcauaccacgaaaaguaccccaccaucuaucaucuucggaagaagcuggucgacuccaccgauaaggccgaucugcgccugaucuacuuggcgcuggcucacaugauuaaguucagaggacacuuucugauagagggcgaccucaaucccgauaacuccgacguggauaagcuguucauccaacuggugcagacguacaaccaacuguuugaagagaauccaaucaacgccagcgggguggacgccaaggccauccuguccgcccggcugucaaaguccagacgccuggagaaucucaucgcgcaacucccuggcgaaaaaaagaacggacucuucgggaaucugauugcucugucccuggggcucacuccgaacuucaagucgaacuucgaccuggcggaggacgcuaagcugcagcuguccaaggacaccuacgaugacgaucuggauaaccuucuggcccagaucggggaucaauacgccgaucucuuccuggccgcaaagaacuugucggaugcuauucugcugagcgacauucugcgggucaauacugaaaucaccaaggcgccccugucggccagcaugaucaagcgcuacgacgaacaccaccaagaccugacucugcugaaggcccucgugcgccagcagcugccugaaaaguacaaggagauuuucuucgaccaguccaagaacggauacgccggauacauugacggaggggccagccaggaggaauuuuacaaauucaucaagcccauucucgagaaaauggacggaaccgaagaguugcucgugaagcugaacagagaggaucuccuccggaagcagcggaccuucgacaacgguuccaucccgcaccaaauccaccugggcgaauugcacgccauccuccggcggcaggaagauuucuacccauucuugaaggacaaucgcgaaaagaucgaaaagaucuugacuuuccgcaucccguacuacgugggcccucuggcccgcggcaacucccgcuucgcuuggaugacacggaaguccgaggaaaccauuacgcccuggaacuucgaggaagugguggacaagggggcguccgcccagagcuucaucgaacgcaugaccaauuucgacaagaaccucccgaacgaaaaagugcugccaaagcacucgcuccucuacgaauacuucaccguguacaacgagcugacuaaggucaaauacgugacugagggaaugcggaagccggccuuccugucgggagagcagaagaaggccauaguggacuugcuuuucaagacuaaccggaaggucacugugaagcaacucaaggaggacuacuucaagaagaucgaguguuucgacucgguggagaucucgggugucgaggaccgcuucaacgccucccugggaacuuaccacgaucugcugaagaucaucaaggacaaggacuuccucgauaacgaagaaaaugaggacauccucgaggauaucgugcugacccugaccuuguucgaggauagggagaugaucgaggagcggcucaagaccuacgcccaccuguuugacgacaaagugaugaagcaacugaaacggcggagguauaccggcuggggucggcugucccgcaagcugaucaacgggaucagggacaagcaguccggaaagaccauccucgacuuccuuaaguccgacggauucgcgaaccgcaacuucaugcaacuuauccacgacgacucgcugacauucaaggaagauauccagaaggcccagguguccggacagggggacucgcuucaugagcacaucgcuaaccuggccggaucccccgccauaaaaaagggcauucugcagaccgucaaagugguggaugagcuggucaaggucaugggccggcauaagccggaaaacaucgucaucgagauggcccgcgagaaccagacuacgcagaagggccagaagaacucccgggagcggaugaagcggauugaagagggcaucaaggagcucggcagccagauucugaaggaacaucccguggaaaacacccagcugcaaaacgaaaagcucuauuuguacuaucugcaaaacggacgcgauauguacguggaucaggagcuggacauuaacagacugagcgacuaugacguggaucacauugugccucaaagcuuccucaaggacgacucaauugacaacaagguccugaccagaagcgacaagaacagaggaaagucggauaaugugccguccgaagaaguggucaagaagaugaagaauuacuggagacagcuccugaaugcgaagcucauuacccagcggaaguucgauaaccugaccaaggccgaaagggguggacuguccgaacucgacaaagcuggcuucaucaagcgccaacuggucgaaaccaggcagaucaccaagcacgucgcccagauucuggacagccgcaugaacacuaaguacgacgagaacgauaagcugauccgcgaagugaaggucaucacccugaaguccaagcucguguccgacuuucggaaggauuuccaguuuuacaagguccgcgagaucaacaacuaccaucacgcccacgacgcguaccuuaacgcagucgugggaacggcucuuaucaagaaguacccaaagcuggagucggaauuuguguacggagacuacaaaguguacgacgugcgcaagaugaucgccaaaucugagcaagagaucgggaaggcaaccgccaaauacuucuucuacucaaacauuaugaauuuuuucaaaacugagauuacccuggcuaacggagaaauucggaagcgcccccugauugaaaccaacggagaaacuggagaaauugugugggacaagggacgggacuucgccaccguccgcaagguccucucaaugccccaagucaacaucgugaaaaagaccgaagugcaaaccggcggcuucucaaaggaguccauccugccuaagcgcaacagcgacaagcugauugccaggaagaaggacugggacccgaagaaguacggaggauuugauuccccuaccguggccuacuccgugcucgugguggccaaaguggaaaaggggaaauccaagaagcugaagucggugaaggagcuuuuggguaucaccaucauggaacgcuccucguucgaaaagaacccaaucgauuuccuggaagcuaaggguuauaaggaagugaaaaaggaccugauuaucaagcugcccaaguacucacuguucgagcuggaaaacggucggaaaaggaugcuggccagcgccggagaacuccagaagggaaacgaacuggcacugccguccaaauacgucaacuuccucuaccuugcaucccauuacgaaaaacucaagggaucgccggaggacaacgagcagaagcagcuuuucguggagcaacacaagcauuacuuggacgagaucaucgagcagauuuccgaguucucaaagcgcgugauccuggccgacgcaaaucuggacaagguccuguccgcguacaauaagcaucgggacaagccuauccgcgaacaggccgagaacaucauccaucuguucacucugacaaaccugggcgcacccgccgcguucaaguacuuugacaccaccaucgauaggaagcgauacaccucaacuaaggaaguguuggacgcgacccuuauccaucagucgaucaccgggcuguacgaaacacggaucgaccucagccaguugggaggcgacaagcgcccugcggcuaccaagaaggccggacaggccaagaagaagaaaugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 47 nt, Artificial Sequence
  Synthetic
aggaaauaagagagaaaagaagaguaagaagaaauauaagagccacc

SEQ ID NO: 16 - DNA, 4444 bp, Artificial Sequence
  Synthetic
aggaaataagagagaaaagaagagtaagaagaaatataagagccaccatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 4444 nt, Artificial Sequence
  Synthetic
aggaaauaagagagaaaagaagaguaagaagaaauauaagagccaccauggccccaaagaagaagcggaaggucgguauccacggagucccagcagccgacaagaaguacagcaucggccuggacaucggcaccaacucugugggcugggccgugaucaccgacgaguacaaggugcccagcaagaaauucaaggugcugggcaacaccgaccggcacagcaucaagaagaaccugaucggagcccugcuguucgacagcggcgaaacagccgaggccacccggcugaagagaaccgccagaagaagauacaccagacggaagaaccggaucugcuaucugcaagagaucuucagcaacgagauggccaagguggacgacagcuucuuccacagacuggaagaguccuuccugguggaagaggacaagaagcacgagagacaccccaucuucggcaacaucguggacgagguggccuaccacgagaaguaccccaccaucuaccaccugagaaagaaacugguggacagcaccgacaaggccgaccugagacugaucuaccuggcccuggcccacaugaucaaguucagaggccacuuccugaucgagggcgaccugaaccccgacaacagcgacguggacaagcuguucauccagcuggugcagaccuacaaccagcuguucgaggaaaaccccaucaacgccagcggcguggacgccaaggcuauccugucugccagacugagcaagagcagaaggcuggaaaaucugaucgcccagcugcccggcgagaagaagaacggccuguucggcaaccugauugcccugagccugggccugacccccaacuucaagagcaacuucgaccuggccgaggaugccaaacugcagcugagcaaggacaccuacgacgacgaccuggacaaccugcuggcccagaucggcgaccaguacgccgaccuguuccuggccgccaagaaccugucugacgccauccugcugagcgacauccugagagugaacaccgagaucaccaaggccccccugagcgccucuaugaucaagagauacgacgagcaccaccaggaccugacccugcugaaagcucucgugcggcagcagcugccugagaaguacaaagaaaucuucuucgaccagagcaagaacggcuacgccggcuacaucgauggcggcgcuagccaggaagaguucuacaaguucaucaagcccauccuggaaaagauggacggcaccgaggaacugcucgugaagcugaacagagaggaccugcugagaaagcagagaaccuucgacaacggcagcaucccccaccagauccaccugggagagcugcacgcuauccugagaaggcaggaagauuuuuacccauuccugaaggacaaccgggaaaagaucgagaagauccugaccuucaggauccccuacuacgugggcccccuggccagaggcaacagcagauucgccuggaugaccagaaagagcgaggaaaccaucacccccuggaacuucgaggaagugguggacaagggcgccagcgcccagagcuucaucgagagaaugacaaacuucgauaagaaccugcccaacgagaaggugcugcccaagcacagccugcuguacgaguacuucaccguguacaacgagcugaccaaagugaaauacgugaccgagggaaugagaaagcccgccuuccugagcggcgagcagaaaaaggccaucguggaccugcuguucaagaccaacagaaaagugaccgugaagcagcugaaagaggacuacuucaagaaaaucgagugcuucgacuccguggaaaucuccggcguggaagauagauucaacgccucccugggcacauaccacgaucugcugaaaauuaucaaggacaaggacuuccuggauaacgaagagaacgaggacauucuggaagauaucgugcugacccugacacuguuugaggaccgcgagaugaucgaggaaaggcugaaaaccuacgcucaccuguucgacgacaaagugaugaagcagcugaagagaaggcgguacaccggcuggggcaggcugagcagaaagcugaucaacggcaucagagacaagcagagcggcaagacaauccuggauuuccugaaguccgacggcuucgccaaccggaacuucaugcagcugauccacgacgacagccugacauucaaagaggacauccagaaagcccagguguccggccagggcgacucucugcacgagcauaucgcuaaccuggccggcagccccgcuaucaagaagggcauccugcagacagugaaggugguggacgagcucgugaaagugaugggcagacacaagcccgagaacaucgugaucgagauggcuagagagaaccagaccacccagaagggacagaagaacucccgcgagaggaugaagagaaucgaagagggcaucaaagagcugggcagccagauccugaaagaacaccccguggaaaacacccagcugcagaacgagaagcuguaccuguacuaccugcagaauggccgggauauguacguggaccaggaacuggacaucaacagacuguccgacuacgauguggaccauaucgugccucagagcuuucugaaggacgacuccaucgauaacaaagugcugacucggagcgacaagaacagaggcaagagcgacaacgugcccuccgaagaggucgugaagaagaugaagaacuacuggcgacagcugcugaacgccaagcugauuacccagaggaaguucgauaaccugaccaaggccgagagaggcggccugagcgagcuggauaaggccggcuucaucaagaggcagcugguggaaaccagacagaucacaaagcacguggcacagauccuggacucccggaugaacacuaaguacgacgaaaacgauaagcugauccgggaagugaaagugaucacccugaaguccaagcugguguccgauuuccggaaggauuuccaguuuuacaaagugcgcgagaucaacaacuaccaccacgcccacgacgccuaccugaacgccgucgugggaaccgcccugaucaaaaaguacccuaagcuggaaagcgaguucguguacggcgacuacaagguguacgacgugcggaagaugaucgccaagagcgagcaggaaaucggcaaggcuaccgccaaguacuucuucuacagcaacaucaugaacuuuuucaagaccgaaaucacccuggccaacggcgagaucagaaagcgcccucugaucgagacaaacggcgaaaccggggagaucgugugggauaagggcagagacuucgccacagugcgaaaggugcugagcaugccccaagugaauaucgugaaaaagaccgaggugcagacaggcggcuucagcaaagagucuauccugcccaagaggaacagcgacaagcugaucgccagaaagaaggacugggaccccaagaaguacggcggcuucgacagcccuaccguggccuacucugugcuggugguggcuaagguggaaaagggcaaguccaagaaacugaagagugugaaagagcugcuggggaucaccaucauggaaagaagcagcuuugagaagaacccuaucgacuuucuggaagccaagggcuacaaagaagugaaaaaggaccugaucaucaagcugccuaaguacucccuguucgagcuggaaaacggcagaaagagaaugcuggccucugccggcgaacugcagaagggaaacgagcuggcccugccuagcaaauaugugaacuuccuguaccuggccucccacuaugagaagcugaagggcagcccugaggacaacgaacagaaacagcuguuuguggaacagcauaagcacuaccuggacgagaucaucgagcagaucagcgaguucuccaagagagugauccuggccgacgccaaucuggacaaggugcugucugccuacaacaagcacagggacaagccuaucagagagcaggccgagaauaucauccaccuguucacccugacaaaccugggcgcuccugccgccuucaaguacuuugacaccaccaucgaccggaagagguacaccagcaccaaagaggugcuggacgccacccugauccaccagagcaucaccggccuguacgagacaagaaucgaccugucucagcugggaggcgacaagagaccugccgccacuaagaaggccggacaggccaaaaagaagaagugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaa